(19) United States
(12) Patent Application Publication
(10) Pub. No.: US 2025/0186622 A1
(43) Pub. Date: Jun. 12, 2025

(54) METHODS FOR NEURAL REGENERATION IN VIVO AND USES THEREOF

(71) Applicant: The Board of Regents of The University of Texas System, Austin, TX (US)

(72) Inventors: Yunjia Zhang, Dallas, TX (US); Chun-Li Zhang, Dallas, TX (US)

(21) Appl. No.: 18/844,042

(22) PCT Filed: Mar. 2, 2023
(86) PCT No.: PCT/US2023/063595

§ 371 Date: Sep. 4, 2024

Related U.S. Application Data
(60) Provisional application No. 63/316,654, filed on Mar. 4, 2022.
(60) Provisional application No. 63/396,025, filed on Aug. 8, 2022.

(51) Int. Cl. A61K 48/00; A61K 9/00; A61K 38/17; A61P 25/28; C12N 15/86

(52) U.S. Cl. CPC A61K 48/0058; A61K 9/0085; A61K 38/1709; A61K 48/0075; A61P 25/28; C12N 15/86; C12N 2740/15043

(57) ABSTRACT

Provided are compositions comprising DLX family transcription factors, and methods for their use for neural regeneration. The compositions can be widely used in regenerative medicine to repair neural injuries or degeneration and improve cell plasticity.

### CROSS REFERENCE TO RELATED APPLICATIONS

This application claims the benefit of the U.S. Provisional application No. 63/316,654 filed Mar. 4, 2022, and the U.S. Provisional application No. 63/396,025 filed Aug. 8, 2022, the disclosures of each which are herein incorporated by reference in their entireties.

### ACKNOWLEDGEMENT OF GOVERNMENT SUPPORT

This invention was made with government support under Grant No. NS099073, NS092616, NS111776, NS117065, and NS088095 awarded by the National Institutes of Health (NIH). The government has certain rights in the invention.

### SEQUENCE LISTING

This application contains a Sequence Listing that has been submitted in xml format via EFS-Web and is hereby incorporated by reference in its entirety. The xml copy is named “106546-750874.xml” and is 60.9 KB in size.

### BACKGROUND

### 1. Field

The present invention relates to medical treatments and applications. In one aspect, the invention relates to neuron regeneration and regenerative medicine.

### 2. Background

During neural development, multipotent neural stem cells (NSCs) sequentially generate neurons and glia that make up the entire central nervous system. Postnatally, these NSCs persist only in discrete regions of the adult mammalian brain, namely the neurogenic niches including the subventricular zone (SVZ) of the lateral ventricle and the subgranular zone (SGZ) of the hippocampus. Neurons generated from the SVZ-NSCs migrate to the olfactory bulb and play a role in olfaction, whereas those from the SGZ remain in the dentate gyrus and participate in learning and memory.

In contrast to region restricted NSCs, the ubiquitously distributed resident glial cells are emerging as a cell source for generation of new neurons through fate reprogramming. Fate reprogramming can be accomplished under certain injury paradigms or through controlling the expression of a single or a combination of fate-determining factors. Induction of new neurons from resident glia has been achieved in multiple non-neurogenic regions, such as the striatum, the cortex, the spinal cord, and the retina.

Nevertheless, a functional neural network requires not only neurons but also glia. Multilineage differentiation will be ideal to provide all three cell types for neural regeneration. This could prove to be fundamentally important for regenerative medicine since neural injuries or degeneration often leads to loss/dysfunction of all three neural lineages. Thus far, such multilineage reprogramming of resident glia has not been possible. There is therefore a need in the field to find agents or factors that can induce multilineage reprogramming of glial cells.

### SUMMARY OF THE INVENTION

The present disclosure is based, at least in part, on the discovery that DLX family transcriptional factors are effective in inducing multilineage differentiation and neural regeneration.

In some aspects the current disclosure encompasses a viral vector comprising a polynucleotide sequence comprising a nucleic acid sequence encoding one or more of a DLX family transcription factor or derivatives, variants or fragment thereof operably linked to a glial cell targeting promoter. In some aspects the viral vector is not an adeno-associated viral vector (AAV). In some aspects examples of suitable viral vectors include but are not restricted to a retrograde virus, retrovirus, herpesvirus, lentivirus, poxvirus, or papiloma virus vector. In some aspects the viral vector is an RNA viral vector. In some aspects the viral vector is a lentiviral vector. In some aspects the glial cell targeting promoter is selected from any one of hGFAP, hgfa2, hALDH1L1, hgfa28, hgfaABC1D hNG2, hIBA1, hCD68, hPDGFRA, or hPDGFRB. In some aspects the polynucleotide sequence further comprises one or more regulatory sequences selected from an enhancer, a leader, a transcription start site (TSS), a linker, 5′ and 3′ untranslated regions (UTRs), an intron, a polyadenylation signal, and a termination region or sequence.

In some aspects the current disclosure encompasses a viral vector comprising a polynucleotide sequence comprising a nucleic acid sequence at least 60% identical to any one of SEQ ID. NOS. 1-6.

In some aspects the current disclosure also encompasses a composition comprising an effective amount of a DLX family transcription factor or derivatives, variants, or fragment thereof, and a pharmaceutically acceptable carrier. In some aspects the composition comprises DLX2.

In some aspects the current disclosure also encompasses a composition comprising an effective amount of an RNA encoding a polypeptide corresponding to a DLX family transcription factor or derivatives, variants or fragment thereof and a pharmaceutically acceptable excipient.

In some aspects the current disclosure encompasses methods for inducing neural regeneration in a subject in need thereof, the method comprising administering to the subject the one or more of the compositions provided herein. In some aspects the subject is a mammal. In some aspects the subject is a human. In some aspect the administering is by any one of an intravenous, intracranial, intrathecal, subcutaneous, or intranasal route.

In some aspects the current disclosure also encompasses a method for inducing multilineage reprogramming of glial cells, the method comprising contacting the cell with a composition comprising the viral vector disclosed herein.

In some aspects the current disclosure encompasses a method for inducing neural regeneration in a subject in need thereof, the method comprising administering to the subject a composition comprising a therapeutically effective amount of a polynucleotide sequence comprising a nucleic acid sequence at least 60% identical to any one of SEQ ID. NOS. 1-6, 13-17. In some aspects the polynucleotide sequence is packaged within a viral vector, wherein the viral vector is not an AAV. In some aspects the viral vector is selected from a group consisting of adenovirus, a retrograde virus, retrovirus, herpesvirus, lentivirus, poxvirus, or papiloma virus. In some aspects the composition further comprises a pharmaceutically acceptable excipient.

In some aspects the subject in need thereof is suspected of, predicted to or diagnosed with a neural injury, or a neurodegenerative disease example of which include neurodegenerative disease Alzheimer's disease, Parkinson's disease, amyotrophic lateral sclerosis, multiple sclerosis, epilepsy, seizures or multiple system atrophy.

In some aspects the current disclosure also encompasses use of the compositions disclosed herein for inducing multilineage neural regeneration in a subject in need thereof and/or for treatment of neural injury, or a neurodegenerative disease.

### BRIEF DESCRIPTION OF THE DRAWINGS

Embodiments of the present inventive concept are illustrated by way of example in which like reference numerals indicate similar elements and in which:

FIG. 1A depicts Images of DCX expression in the adult striata injected with viruses expressing the indicated genes. wk, week; IHC, immunohistochemistry. Scales: 1 mm for lower magnification views and 20 μm for zoom view.

FIG. 1B is a bar graph showing the quantifications of the number of induced DCX+ cells/injection (n=2-4 mice per condition).

FIG. 1C is a bar graph showing a time course analysis of DLX2-induced DCX+ cells (n=3; p=0.0083 and F (3,8)=8.101 by one-way ANOVA).

FIG. 1D shows a schematic of the study design to analyze reprogramming efficiency (top), confocal images showing co-expression of DCX and GFP (Scales: 20 μm, bottom left) and quantification of DLX2-mediated reprogramming efficiency (n=2,448 GFP+ cells from 3 mice, bottom right).

FIG. 1E shows a schematic of the study design to examine cell proliferation with BrdU incorporation (top), confocal images showing BrdU-labeled DCX+ cells. Scales: 20 μm (bottom left) and quantification of BrdU-labeled DCX+ cells (n=230 DCX+ cells from 4 mice) (bottom right)

FIG. 1F shows a schematic of the study design to trace adult astrocytes and their derivatives Tam, tamoxifen (top), confocal images of astrocyte-derived DCX+ cells indicated by tdT. Scales: 20 μm (bottom left) and quantification of astrocyte-derived DCX+ cells (n=421 DCX+ cells from 3 mice) (bottom right).

FIG. 1G shows a schematic of the study design to trace NG2 glia and their derivatives (top), confocal images showing absence of the YFP reporter in DCX+ cells. Scales: 20 μm. (bottom left), bar graph showing that DLX2-induced DCX+ cells do not come from NG2 glia (n=384 DCX+ cells from 2 mice) (bottom right).

FIG. 1H shows a schematic of the study design to trace endogenous NSCs and their derivatives (top), a bar graph showing that DLX2-induced DCX+ cells do not come from endogenous NSCs (n=180 DCX+ cells for LV and n=350 DCX+ cells for Stm from 2 mice) LV, lateral ventricle; Stm, striatum (middle) and confocal images showing expression of DCX and the tdT reporter in the indicated brain regions. tdT+ cells are not present in the adult striatum. Scales: 200 μm for upper panels and 20 μm for lower panels (bottom).

FIG. 2 depicts confocal images showing how DCX+ cells were quantified. DCX+ cells were identified by staining of DCX and HST (yellow asterisks) through a z-series of 1-μm thick confocal images. Scale: 20 μm.

FIG. 3 depicts expression of DLX2 and GFP during the reprogramming process. The study design for a time-course analysis of DLX2 expression and the co-expressed GFP post viral injections is shown on top andconfocal images at the indicated time points are provided a the bottom. DLX2 is rapidly downregulated, while GFP is relatively stable for an extended time-period. DCX+ cells are induced by DLX2. Scale: 20 μm.

FIG. 4 depicts the identity of BrdU-labeled cells in the adult mouse striatum. The left two panels shows that OLIG2+NG2 glia and IBA1+ microglia are the major cells labeled by BrdU in the non-injected contralateral striatum. Mice were treated as in FIG. 1E. Scale: 20 μm. BrdU+ cells include OLIG2+NG2 glia, IBA1+ microglia and SOX9+ astrocytes in the DLX2 virus-injected striatum (right panel). Mice were treated as in FIG. 1E. Scale: 20 μm.

FIG. 5A depicts the genetic lineage tracing of adult astrocytes or NG2 glia. Top left provides a schematic of the study design to trace adult astrocytes. The bar graph in the top middle panel shows that astrocytes are specifically traced in Aldh1I1-CreERT2; R26R-tdT mice (n=339 tdT+ cells for ALDH1L1, n=553 tdT+ cells for ALDOC, n=197 tdT+ cells for APC, n=442 tdT+ cells for NeuN, n=511 tdT+ cells for NG2, and n=272 tdT+ cells for IBA1 from 2-3 mice). The bar graph in the top right shows the astrocyte labeling efficiency in Aldh1I1-CreERT2; R26R-tdT mice (n=174 cells for ALDH1L1 and n=626 cells for ALDOC from 3 mice). Confocal images of tdT-traced cells in Aldh1I1-CreERT2; R26R-tdT mice. Scales: 20 μm are provided in the bottom panels.

FIG. 5B shows a schematic of the study design to trace adult NG2 glia (top left), NG2 glia are specifically traced in Pdgfra-CreERT2; R26R-YFP mice (n=224 YFP+ cells for NG2, n=206 YFP+ cells for NeuN, and n=348 YFP+ cells for ALDOC from 2 mice) as seen from the bar garphConfocal images of YFP-traced cells in Pdgfra-CreERT2; R26R-YFP mice are provided. Scales: 20 μm.

FIG. 6 depicts confocal images showing how DCX+ tdT+ cells were quantified. Cells were identified by staining of DCX, tdT and HST through a z-series of 1-μm thick confocal images. Scale: 20 μm.

FIG. 7A depicts the reprogramed astrocytes becoming ASCL1+ neural progenitor cells. The top right shows a schematic of the study design to analyze DLX2-induced ASCL1+ cells. A bar graph showing the time-course analysis of DLX2-induced ASCL1+ cells (n=3 mice per time point) is provided along with confocal images of DLX2-induced ASCL1+ cells at the indicated time points. Scales: 20 μm.

FIG. 7B shows the study design to trace DLX2-induced ASCL1+ cells and their derivatives, bar graph showing the quantification of DLX2-induced DCX+ cells transiting through an ASCL1+ stage (n=365 DCX+ cells from 3 mice). (F) Confocal images of ASCL1-derived DCX+ cells indicated by tdT. tdT+ or DCX+ cells were not detected in the control (Ctrl) virus-injected striatum. Scales: 20 μm.

FIG. 7C shows the study design to analyze the timing of astrocyte-reprogramming and bar graph showing the quick loss of astrocyte-identity when reprogrammed by DLX2. Quantification was conducted at 4 wpv (n=463 DCX+ cells from 3 mice). Representative confocal images of lineage-traced cells showing a quick loss of astrocyte-identity when reprogrammed are provdide. Enlarged views of the boxed regions are shown on the right panels. Scales: 20 μm. A schematic summary of the reprogramming process is shown in the bottom panel.

FIG. 8 depicts lineage progression of DLX2-mediated reprogramming. A schematic of the study design to examine astrocyte reprogramming by ectopic DLX2 is provided. Confocal images showing ASCL1+ iNPCs are derived from tdT-traced astrocytes. Scale: 20 μm and bar graph showing the quantification data of tdT-traced ASCL1+ iNPCs (n=149 ASCL1+ cells from 2 mice) and confocal images showing transitional cell states from tdT-traced astrocytes to ASCL1+ iNPCs to DCX+ neuroblasts. 20 μm, are provided. Also shown is a schematic summary of lineage progression of DLX2-reprogrammed astrocytes.

FIG. 9 depicts the effect of neurotrophic factors on maturation of DLX2-induced neurons. A schematic of the study design to examine DLX2-induced neurons-DLX2-induced iNPCs and their derivatives were traced in Ascl1-CreERT2; R26R-tdT mice after virus injections and Tam treatments. iNPC-derived neurons were examined at 8 wpv. Bar graph showing the quantification of iNPC-derived neurons under the indicated conditions and confocal images of iNPC-derived NeuN+ neurons under the indicated conditions. Of note, tdT+ cells were not induced under the control BDNF-NOG condition, Scale: 20 μm are provided.

FIG. 10 depicts confocal images of BrdU-labeled neurons at 8 wpv. Adult mice were injected with lentivirus expressing BDNF or DLX2 and BDNF. They were then supplied with BrdU in drinking water for 4 weeks and analyzed at 8 wpv. Examples of BrdU+ NeuN+ cells are indicated by arrows. Scale: 20 μm.

FIG. 11A depict the reprogramed astrocytes generate mature neurons. The study design to analyze DLX2-induced neurons, confocal images of DLX2-induced neurons with complex morphology at 12 wpv. Scale: 20 μm and bar graphs showing the Quantification of neuronal subtypes (n=3-4 mice; n=451 NeuN⁺tdT⁺ cells for GABA+NeuN⁺tdT⁺, CTIP2+NeuN⁺tdT⁺ and CR+NeuN⁺tdT⁺; n=6 brain slices for each of the other markers; n=594 tdT+ cells for CR+NeuN-tdT+ cells) are provided.

FIG. 11B shows representative confocal images showing DLX2-induced GABAergic neurons. Scale: 20 μm (top panel), confocal images showing DLX2-induced CTIP2+ neurons. Scale: 20 μm (2^{nd} panel), Confocal images showing DLX2-induced CR+ neurons. 3rd panel: NeuN+CR+ neurons; 4th panel: NeuN-CR+ neurons. Scale: 20 μm and confocal images showing DLX2-induced striatal-like neurons, 5^{th} panel: DARPP32+CTIP2-neurons; 6th panel:: DARPP32-CTIP2+ neurons. Scale: 20 μm.

FIG. 12 depicts that no direct induction of CTIP2 expression by ectopic DLX2 was seen. A schematic of the study design showing that adult mice were injected with DLX2 virus and examined at 1 or 2 wpv and representative confocal images showing a lack of colocalization of DLX2 and CTIP2 are provided. Scales: 20 μm.

FIG. 13 depicts the promoted generation of astrocytes. A schematic of the study design to determine the effect of DLX2 on the number of astrocytes, bar graphs showing quantification of SOX9+ astrocytes surrounding the virus-injected regions (p=0.0021, F (2,6)=20.34 by one-way ANOVA) and representative confocal images of SOX9+ astrocytes surrounding the virus-injected regions are provided. Scale: 20 μm.

FIG. 14A depict the astrocyte-converted ASCL1+ cells are multipotent NPCs. A schematic showing the study design for fate analysis of DLX2-induced ASCL1+ iNPCs, bar graphs showing the quantification of cell types derived from iNPCs at the indicated time points (n=5,487 tdT+ cells from 3 mice for 4 wpv, n=7,982 tdT+ cells from 3-4 mice for 8 wpv, and n=3,665 tdT+ cells from 3-4 mice for 12 wpv) and representative confocal images of multiple cell types derived from DLX2-induced ASCL1+ iNPCs at 12 wpv are provided. Scales: 20 μm

FIG. 14B provides representative confocal images of proliferating iNPCs at 12 wpv. a, an enlarged view of proliferating iNPCs in the boxed region a; b, an enlarged view of iNPC-derived, non-proliferative astrocyte in the boxed region b. Scales: 20 μm, bar garphs showing the quantification of proliferating iNPCs at 12 wpv (n=309 tdT+ cells from 3 mice) and a schematic summary of the reprogramming process and cell identities.

FIG. 15 depicts that iNPCs generate OPCs and oligodendrocytes. A schematic of the study design to examine DLX2-induced OPCs and oligodendrocytes, representative confocal images showing DLX2-induced SOX10+ OLIG2+ tdT+ OPCs at 4wpv (bottom left) and representative confocal images showing DLX2-induced SOX10+ OLIG2+ tdT+ OPCs and MBP+ OLIG2+ tdT+ oligodendrocytes at 12 wpv are provided. Scales: 20 μm.

FIG. 16 depict the isolation of single cells through fluorescence activated sorting. A schematic of the study design to isolate traced astrocytes and their derivatives and FACS data with striatal tissues from wildtype mice were used as negative controls for FACS are provided.

FIG. 17A depict the scRNA-seq analysis of DLX2-mediated reprogramming. A schematic of the workflow of scRNA-seq experiment is provided. tdT+ striatal cells were sorted from adult Aldh1I1-CreERT2; R26R-tdT mice after Tam treatments and virus injections (n=5 for Lenti-DLX2 group and n=4 for Lenti-GFP control group). Dissociated single tdT+ cells were then processed for scRNA-seq. The panel of 31 known marker genes used for clustering cell types and cell type distributions between Lenti-DLX2 and Lenti-GFP samples are shown.

FIG. 17B shows the distributions of neural cell types within the Lenti-DLX2 sample. Decreased Gfap expression was seen along the reprogramming process. Gene expression is measured in In (readcount+1). Also shown is enriched expression of the cell cycle genes Cdk1 and Mki67 in cycling NPC cluster, ascl1 expression in both NPC clusters, high Dcx expression in NPC and neuroblast clusters, restricted expression of the mature GABAergic marker Calb2 in late neuroblasts. (K-L) Olig1 and Olig2 expression in NPCs, cluster 5 genes (categorized as neurogenesis genes) are highly enriched for putative target genes of Dlx1/2. p=7.37e-35 (Chi-square test). Genes in reconstructed Dlx1 regulon is highly enriched for putative target genes of Dlx1/2 (Lindtner et al., 2019) as seen in the bottom 3 panels: putative target genes from RNA-seq in published data (Lindtner et al., 2019); p=4.02e-45 (Chi-square test) and genes associated with chromatin activation by DLX1/2 from ChIPseq data in published data (Lindtner et al., 2019); p=6.13e-40 (Chi-square test) is shown.

FIG. 18A depicts gene programs and regulatory networks underlying DLX2-induced reprogramming of adult astrocytes. Lenti-DLX2 and Lenti-GFP control cells are clustered. 18 neural and non-neural cell types are identified by examining the expression of known marker genes, and 2 clusters could not be identified by these genes. A putative reprogramming trajectory (grey dashed box) consisting of astrocytes, lenti-astrocytes, NPCs, and neuroblasts is further examined. A panel of marker genes are used to identify astrocytes, NPCs, neuroblast, NG2 glia, and oligodendrocytes. Sub-clustering of the putative reprogramming trajectory from astrocytes to lenti-astrocytes, NPCs, and neuroblasts reveals heterogeneity within each cell type. Putative reprogramming trajectory is indicated by dark purple arrow. Astrocyte lineage tracer tdT (top) and lentiviral transcript (middle) are expressed along the reprogramming trajectory. The trajectory consists of mainly Lenti-DLX2, along with a few Lenti-GFP cells (bottom). (E) Diffusion pseudotime is calculated. Cells are then ranked based on their pseudotime (low rank=early pseudotime).

FIG. 18B provides unsupervised clustering data identifying 12 clusters of 2,584 highly variable genes showing five major expression patterns along pseudotime: astrocyte genes (clusters 6 and 0), lenti-astrocyte genes (clusters 2, 10), transition genes (cluster 3), NPC genes (clusters 9, 7, and 1), and neurogenesis genes (clusters 4, 8, 5, and 11). Gene ontology terms enriched in each gene cluster highlight the associated biological processes. Gene regulatory network analysis identifies potential regulators of each stage of reprogramming.

FIG. 19 depicts a tracing of non-neural cells in Aldh1I1-CreERT2; R26R-tdT mice after virus injections. A schematic of the study design and Confocal images of the indicated markers is shown. Some non-neural cells could be traced by tdT in the virus-injected regions. Scales: 20 μm.

FIG. 20A depicts reprogramming of astrocytes by DLX2-induced regulons. (A) Study design to examine reprogramming activity of DLXs. (B) Quantification of DLX-induced DCX+ cells (n=4-5 mice per condition). (C) Low magnification images of DLX-induced DCX+ cells. Scale: 500 μm.

FIG. 20B shows a schematic of study design to trace adult astrocytes and their derivatives. Bar graphs showing the quantification of astrocyte-derived DCX+ cells (DLX5: n=323 DCX+ cells from 3 mice; DLX1: n=133 DCX+ cells from 2 mice) are representative confocal images of astrocyte-derived DCX+ cells as indicated by tdT are provided. Scales: 20 μm.

FIG. 20C shows a schematic of the study design to label proliferating cells with BrdU. The bar graph shows the quantitation of the proliferative intermediate detected by BrdU-incorporation for DLX-induced DCX+ cells (DLX1: n=304 DCX+ cells from 3 mice; DLX5: n=584 DCX+ cells from 3 mice) and representative confocal images showing BrdU-labeled DCX+ cells are provided. Scales: 20 μm.

FIG. 21 depicts the effect of downregulation of endogenous Dlx1 and/or Dlx5 on DLX2-mediated reprogramming. A western blott analysis of shRNA-mediated knockdown is shown (top left). HA-tagged mouse Dlx1 or Dlx5 were co-expressed with the indicated shRNAs. A shRNA against luciferase was used as the control (Ctrl). Protein expression was determined by western blots with an anti-HA antibody. ACTB was used as the loading control. A schematic of the study design to determine the effect of shRNAs on DLX2-mediated reprogramming, bar graph showing that downregulation of either Dlx1, Dlx5, or their combination had minimal effect on DLX2-induced neurons (p=0.4939 and F (3,10)=0.8582 by one-way ANOVA) and a low magnification view of DLX2-induced DCX+ cells under the indicated conditions is provided. Scale: 1 mm.

FIG. 22A depict DLX2-mediated reprogramming requires suppression of Notch signaling. A schematic of the study design to determine the role of Notch signaling is provided. Low magnification views of DLX2-induced DCX+ cells under the indicated conditions (Scale: 1 mm) and quantifications showing that constitutive Notch signaling suppressed DLX2-induced DCX+ cells (p=0.0286 by two-tailed t-test) are provided.

FIG. 22B provides a schematic showing the study design to examine expression of DLX2 and ASCL1, representative sonfocal images showing co-localization of DLX2 and ASCL1 within the same cells (Scale: 20 μm) and bar graph showing the and quantifications showing a higher number of ASCL1+ cells also expressed DLX2 at 2 wpv (n=217 ASCL1+ cells from 3 mice) are provided.

FIG. 23A depicts the co-clustering of reprogrammed cells with WT E18.5 brain cells. The single cells from the WT E18.5 and the DLX2-induced reprogramming datasets have comparable captured transcript complexity and number of genes detected. Similar to FIG. 5A, neural and non-neural clusters are identified by the expression of a panel of marker genes. Cell type distributions among the source samples is provided.

FIG. 23B provides ranking of cell clusters in FIG. 6A based on diffusion pseudotime. Repression and activation of genes involved in adult hippocampal neurogenesis along pseudotime are shown. Comparing to WT E18.5 cells, Lenti-DLX2 cells exhibit largely similar changes, but with more abrupt changes of genes involved in NSC quiescence and activation (especially Apoe and adhoc).

FIG. 24A depicts scRNA-seq reveals NSC-like behaviors of reprogrammed astrocytes. Lenti-DLX2 and Lenti-GFP control cells are co-clustered with a WT E18.5 mouse brain dataset. A subclustering of the main trajectory (i.e. astrocytes, lenti-astrocytes, NPCs, and neuroblasts) is shown. A UMAP clustering of all cell types was performed. The trajectory consists of mainly WT E18.5 and Lenti-DLX2, but not Lenti-GFP cells (top left). As expected, astrocyte lineage tracer tdT (bottom left) and lentiviral transcript (top right) are expressed along the reprogramming trajectory. Rank of inferred pseudotime (bottom right) for the reprogramming trajectory is calculated (low rank=early pseudotime). In Lenti-DLX2 cells, known marker genes during adult neurogenesis in SVZ are activated or repressed in the expected order along pseudotime, like the WT E18.5 cells (right). Differential expression analysis is performed between Lenti-DLX2 and WT E18.5 cells at each stage of the trajectory. Though different in the first half of the trajectory, reprogramming and neurogenesis converge towards the end (left). Two key differences are the expression of cell cycle and immune response genes (middle and right). The trajectory consists of mainly WT E18.5 and Lenti-DLX2, but not Lenti-GFP cells (top left). As expected, astrocyte lineage tracer tdT (bottom left) and lentiviral transcript (top right) are expressed along the reprogramming trajectory. Rank of inferred pseudotime (bottom right) for the reprogramming trajectory is calculated (low rank=early pseudotime).

FIG. 24B shows that in Lenti-DLX2 cells (left), known marker genes during adult neurogenesis in SVZ are activated or repressed in the expected order along pseudotime, like the WT E18.5 cells (right). Differential expression analysis is performed between Lenti-DLX2 and WT E18.5 cells at each stage of the trajectory. Though different in the first half of the trajectory, reprogramming and neurogenesis converge towards the end (left). Two key differences are the expression of cell cycle and immune response genes (middle and right).

FIG. 25A depict the co-clustering of reprogrammed cells with adult hippocampal brain cells. Clustering of the putative reprogramming/neurogenesis trajectory reflecting adult neurogenesis from astrocytes/qNSCs, to radial glia-like (RGL) cells, neuronal intermediate progenitor cells (nIPCs), and granule cells (GCs) is shown in the left panel. Clustering map of single cells, with cells colored by their original annotation in Hochgerner et al., 2018 (top right). Adult neurogenesis cells align well with DLX2-induced reprogramming, mingling in the astrocyte NPC/nIPC states.

FIG. 25B shows diffusion pseudotime as calculated. Cells are then ranked based on their pseudotime (low rank=early pseudotime). In adult neurogenesis, known marker genes during adult neurogenesis in SVZ (from Dulken et al., 2017) are activated or repressed in the expected order along pseudotime, more similar to Lenti-DLX2 cells than WT E18.5 cells. In adult neurogenesis, known marker genes during adult neurogenesis in hippocampus (from Shin et al., 2015) are activated or repressed in the expected order along pseudotime, more similar to Lenti-DLX2 cells than WT E18.5 cells.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized, and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based on, in part, the surprising discovery that expression of DLX family transcription factors, such as DLX2, can unleash the multipotentiality of adult resident astrocytes. DLX enables astrocytes to rapidly become neural progenitor cells, which give rise to neurons, astrocytes, and oligodendrocytes. Further, the reprogrammed astrocytes have a neural stem cell-like behavior, such as transitioning from quiescence to activation, proliferation, and neurogenesis. Thus, activation of DLX family transcription factors and suppression of Notch signaling are candidates for neural regeneration, and/or repairing injured neurons.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The terms “nucleic acid”, “nucleic acid molecule”, and “polynucleotide” are used interchangeably herein. The terms “nucleic acid encoding”, or “nucleic acid molecule encoding” should be understood as referring to the sequence of nucleotides which encodes a polypeptide.

A polynucleotide described herein may comprise one or more nucleic acids each encoding a polypeptide, operably linked to (i.e., in a functional relationship with) one or more regulatory sequences, such as a promoter. Such a polynucleotide may alternatively be referred to herein as a “nucleic acid construct” or “construct”.

As used herein, “regulatory elements” or “control elements” refer to any sequence elements that regulate, positively or negatively, the expression of an operably linked sequence. “Regulatory elements” include, without being limiting, a promoter, an enhancer, a leader, a transcription start site (TSS), a linker, 5′ and 3′ untranslated regions (UTRs), an intron, a polyadenylation signal, internal ribosome entry sites (IRES), splice junctions, and a termination region or sequence, etc., that are suitable, necessary or preferred for regulating or allowing expression of the gene or transcribable DNA sequence in a cell. Such additional regulatory element(s) can be optional and used to enhance or optimize expression of the gene or transcribable DNA sequence. A regulatory sequence can, for example, be inducible, non-inducible, constitutive, cell-cycle regulated, metabolically regulated, and the like. A regulatory sequence may be a promoter. As used herein, the term “promoter” refers to a DNA sequence that contains an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein. In some exemplary aspects the promoter sequence is adapted to enable expression of a polynucleotide in glial cells. Non-limiting examples of suitable promoters include hGFAP, hgfa2, hALDH1L1, hgfa28, hgfaABC1D, hNG2, hIBA1, hCD68, hPDGFRA, hPDGFRB. As used herein, the term “enhancer” refers to a region of DNA sequence that operates to initiate, assist, affect, cause, and/or promote the transcription and expression of the associated transcribable DNA sequence or coding sequence, at least in certain tissue(s), developmental stage(s) and/or condition(s). In an aspect, an enhancer is a cis enhancer. In one aspect, an enhancer is a trans enhancer.

As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s).

Vector: A vector may comprise a nucleic acid construct or an expression construct as earlier defined herein. A vector as described herein may be selected from any genetic element known in the art which can facilitate transfer of nucleic acids between cells, such as, but not limited to, plasmids, transposons, cosmids, chromosomes, artificial chromosomes, viruses, virions, and the like. A vector may also be a chemical vector, such as a lipid complex or naked DNA. “Naked DNA” or “naked nucleic acid” refers to a nucleic acid molecule that is not contained in encapsulating means that facilitates delivery of a nucleic acid into the cytoplasm of a target host cell. Naked DNA may be circular or linear (linearized DNA sequence). Optionally, a naked nucleic acid can be associated with standard means used in the art for facilitating its delivery of the nucleic acid to the target host cell, for example to facilitate the transport of the nucleic acid through the cell membrane. A vector may be a viral vector and/or a gene therapy vector.

A viral vector and/or a gene therapy vector may be any viral vector known in the art. These vectors may comprise a polynucleotide as disclosed herein operably linked to a glial targeting promoter.

A gene therapy vector is a vector that is suitable for gene therapy. Vectors that are suitable for gene therapy are described in Anderson 1998, Nature 392:25-30; Walther and Stein, 2000, Drugs 60:249-71; Kay et al., 2001, Nat. Med. 7:33-40; Russell, 2000, J. Gen. Virol. 81:2573-604; Amado and Chen, 1999, Science 285:674-6; Federico, 1999, Curr. Opin. Biotechnol. 10:448-53; Vigna and Naldini, 2000, J. Gene Med. 2:308-16; Marin et al., 1997, Mol. Med. Today 3:396-403; Peng and Russell, 1999, Curr. Opin. Biotechnol. 10:454-7; Sommerfelt, 1999, J. Gen. Virol. 80:3049-64; Reiser, 2000, Gene Ther. 7:910-3; and references cited therein.

“Transduction” refers to the delivery of a polynucleotide as disclosed herein to a glial cell using a viral vector. For example, transduction of a cell by a retroviral or lentiviral vector of the invention leads to transfer of the genome contained in that vector into the transduced cell. In an aspect, the vector is a lentiviral vector.

A “genetically modified” or “modified cell” refers to a cell in which the nuclear, organellar or extrachromosomal nucleic acid sequences of a cell has been transformed, modified or transduced using recombinant DNA technology to comprise a heterologous nucleic acid molecule, and is used interchangeably with “engineered cell,” “transformed cell,” and “transduced cell.”

### II. Compositions

### (a) Polynucleotides

The Distal-Less Homeobox (DLX) family, including DLX1, DLX2, DLX3, DLX5 and DLX6, are transcriptional regulators regulating forebrain and craniofacial development. The present invention shows that DLX family transcriptional factors, such as DLX2, when expressed in adult resident glial cells, can unleash the multipotentiality of the cells. In some exemplary aspects described herein DLX2 expression in astrocytes, enables astrocytes to rapidly become neural progenitor cells, which give rise to neurons, astrocytes, and oligodendrocytes.

In some aspects the current disclosure encompasses isolated polynucleotide sequence comprising a nucleic acid sequence encoding one or more of a DLX family transcription factor or variants, derivatives or fragments thereof, operably linked to a glial cell targeting promoter. In some aspects the polynucleotide may comprise a nucleic acid sequence corresponding to any one of a mammalian DLX family gene including but not restricted to DLX1, DLX2, DLX3, DLX4, DLX5 or DLX6 or any derivative, variant or fragment thereof, capable of encoding a functional DLX polypeptide. In some aspects the DLX family gene sequence is a human gene sequence or variants, derivative or fragments thereof. In some aspects the gene sequence is a non-human gene sequence or variants, derivative or fragments thereof optimized to express in a mammalian cell, for example a human cell.

In some exemplary aspects the current disclosure encompasses polynucleotides comprising a nucleic acid sequence at least 60% similar or identical to any one of SEQ ID NOS: 1-6 or variants, derivative or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In some aspects the nucleic acid sequence provided herein encodes for an amino acid sequence at least 60% identical to any one of SEQ ID NOS: 7-12 as provided in Table A or variants, derivative or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In some aspects the polynucleotide may further comprise a nucleic acid comprising a IRES sequence.

In some aspects the polynucleotide disclosed herein may also be an RNA sequence encoding for an amino acid sequence at least 60% identical to any one of SEQ ID NOS: 7-12 or variants, derivative or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

### (b) Vectors

In some aspects the current disclosure also encompasses vectors that facilitate transfer of nucleic acids encoding the disclosed DLX transgene to cells, such as, but not limited to, plasmids, transposons, cosmids, chromosomes, artificial chromosomes, viruses, virions, and the like. A vector may also be a chemical vector, such as a lipid complex or naked DNA. In some aspects the vector comprises one or more polynucleotide sequences provided herein. In some exemplary aspects the vector comprises a polynucleotide comprising a nucleic acid sequence at least 60% similar or identical to any one of SEQ ID NOS: 1-6 as provided in Table A or variants, derivative or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%. In some aspects the nucleic acid sequence encodes an amino acid sequence at least 60% identical to any one of SEQ ID NOS: 7-12 as provided in Table A or variants, derivative or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In some aspects the vector may be a viral vector as provided in the definitions provided herein. A viral vector and/or a gene therapy vector may be any viral vector known in the art. These vectors may comprise a nucleic acid molecule or nucleic acid construct as described herein. In some aspects the viral vector comprises one or more polynucleotide sequences provided herein. In some exemplary aspects the vector comprises a polynucleotide comprising a nucleic acid sequence at least 60% similar or identical to any one of SEQ ID NOS: 1-6 as provided in Table A or variants, derivative or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%. In some aspects the nucleic acid sequence encodes an amino acid sequence at least 60% identical to any one of SEQ ID NOS: 7-12 as provided in Table A or variants, derivative or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

A suitable vector includes a retroviral vector. A preferred retroviral vector for application in the present invention is a lentiviral based viral vector. Lentiviral vectors have the ability to infect and to stably integrate into the genome of dividing and non-dividing cells (Amado and Chen, 1999 Science 285:674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10:448-53) and Vigna et al. (2000, J Gene Med 2000; 2:308-16).

In an aspect, the vector is a viral vector, preferably a lentiviral vector. In some aspects the lentiviral vector as available in the art or known in the art and derivatives thereof, further comprising the polynucleotides provided herein. In some exemplary aspects the lentiviral vector comprises a sequence at 60% identical to SEQ ID NOS. 13-16.

Other suitable viral and/or gene therapy vectors include a herpes virus vector, a polyoma virus vector or a vaccinia virus vector. In some particular aspects the viral vector is not an Adeno Associated viral vector.

A viral and/or gene therapy vector comprises a nucleotide encoding a DLX gene whereby each of said nucleotide sequence is operably linked to the appropriate regulatory sequences. Such regulatory sequence will at least comprise a promoter sequence. Suitable promoters for expression of such a nucleotide sequence promote expression of the transgene in the transduced astrocytes. Exemplary promoter sequences include but are not restricted to hGFAP, hgfa2, hALDH1L1, hgfa28, hgfaABC₁D, hNG2, hIBA1, hCD68, hPDGFRA, hPDGFRB. In some aspects, the promoter sequence comprises a nucleic acid sequence at least 60% similar or identical to any one of SEQ ID NOS: 17. In some aspects the vector may further comprises other regulatory elements. In some aspects the regulatory signal is an enhancer sequence. By “enhancer” is meant a nucleic acid sequence that, when positioned proximate to a promoter, confers increased transcription activity relative to the transcription activity resulting from the promoter in the absence of the enhancer domain. Non-limiting examples of enhancers include CMV enhancer, MIE enhancer, GADD45G, HACNS1. In some aspect viral vector may comprise a viral posttranscriptional regulatory element. In some embodiments, the viral posttranscriptional regulatory element is woodchuck hepatitis virus posttranscriptional regulatory element (WPRE), hepatitis B virus posttranscriptional regulatory element (HBVPRE), RNA transport element (RTE), or any variant thereof. In some aspects the viral vector may comprise a transcription termination region downstream of the posttranscriptional regulatory element. In some embodiments, the transcription termination region comprises an SV40 late poly(A) sequence, a rabbit beta-globin poly(A) sequence, a bovine growth hormone poly(A) sequence, or any variant thereof. Transposon or other non-viral delivery systems may also be used in this context. All systems can be used in vitro, ex vivo or in vivo.

A viral and/or gene therapy vector may optionally comprise a further nucleotide sequence coding for a further polypeptide. A further polypeptide may be a (selectable) marker polypeptide that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Green (supra).

In some aspects the vector or viral vectors provided in the current disclosure can be transduced or transfected into a suitable host cell.

### (c) Polypeptides

In some aspects the current disclosure also encompasses functional DLX transcription family proteins or derivatives, variants or fragments thereof that may be used in compositions to trigger multipotential differentiation of astrocytes. In some aspects the polypeptides comprise an amino acid sequence an amino acid sequence at least 60% identical to any one of SEQ ID NOS: 7-12 as provided in Table A or variants, derivative, or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In some aspects the polypeptides of the current disclosure may comprise one of more of a signal peptide, leader sequence, a cleavage site, a purification tag, 2A self-cleaving peptide, modified amino acids. In some aspects the polypeptides of the current disclosure may comprise one or more additional polypeptide sequences as a fusion protein. These include but are not restricted to fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene.

### (d) Host and Engineered Cell

In some aspects the current disclosure also encompasses host cell comprising one or more of the polynucleotides, vectors, viral vectors, polypeptides disclosed herein. In some aspects the host cell is adapted to maintain, replicate, propagate express and/or increase the copy number or load of the polynucleotide, vectors or viral vector of the current disclosure. Examples of workable combinations of cell lines and expression vectors are described in Sambrook and Russell (2001, supra) and in Metzger et al. (1988) Nature 334:31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media.

In some aspects the host cell is adapted to express the polypeptides disclosed herein. In some aspects the host cell is a cell that is capable of undergoing multilineage reprogramming on expression of the polypeptides disclosed herein. A host cell may be a cell that is suitable for culture in liquid or on solid media. In some aspects the host cell is a mammalian cell. In some aspects the host cell is a human cell. In some exemplary aspect the host cell is NG2 glia, fibroblasts, microglia or neural progenitor cells. In some aspects the host cell is an astrocyte. In some aspects the host cell is an isolated cell. In some aspects the host cell may exist in vitro, ex vivo or in vivo.

In some aspects the host cell is an engineered or genetically modified cell. In one aspect, a genetically modified cell as disclosed herein expresses a protein encoded by a nucleic acid molecule engineered in such manner to contain an insertion of at least one nucleotide, a deletion of at least one nucleotide, and/or a substitution of at least one nucleotide in a sequence encoding at least one heterologous protein. “Engineered cells” refers herein to cells having been engineered, e.g., by the introduction of an exogenous nucleic acid sequence as defined herein. Such a cell has been genetically modified for example by the introduction of for example one or more mutations, insertions and/or deletions in the endogenous gene and/or insertion of a genetic construct in the genome. The modification may have been introduced using recombinant DNA technology. An engineered cell may refer to a cell in isolation or in culture. Engineered cells may be “transduced cells” wherein the cells have been infected with e.g., a modified virus, for example, a retrovirus may be used but other suitable viruses may also be contemplated such as lentiviruses. Non-viral methods may also be used, such as transfections. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient but may also be a stable transfection wherein cells can be selected that have the gene construct integrated in their genome. In some cases, genetic engineering systems such as CRISPR or Argonaute may be utilized to design engineered cells that express a polypeptide described herein. A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems. A CRISPR system can be utilized to facilitate insertion of a polynucleotide sequence encoding a protein or a component thereof into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA. In some aspects the current disclosure also encompasses compositions for example a CRISPR/Cas system adapted to generate an engineered glial cell comprising the polynucleotides disclosed herein. In some aspects the CRISPR/Cas system may be delivered as a polynucleotide or encoded in of a viral vector.

In some aspects the engineered host cell comprises a polynucleotide or a polypeptide disclosed herein. In some aspects the engineered host cell may be an in vitro, ex vivo or in vivo cell. In some aspects the engineered cells is genetically modified to comprise a polynucleotide sequence at least 60% similar or identical to any one of SEQ ID NOS: 1-6 as provided in Table A or variants, derivative or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%. In some aspects the host cell comprises a polypeptide with an amino acid sequence at least 60% identical or similar to any one of SEQ ID NOS: 7-12 as provided in Table A or variants, derivative or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

### (e) Pharmaceutical Formulations

In certain embodiments, the composition disclosed herein may be provided per se or as part of a pharmaceutical composition. In some aspects one or more of the polynucleotides, viral vectors, host cell or the DLX family transcription factor(s) disclosed herein can be mixed with suitable carriers or excipients and provided as a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the peptide, antibody, chemical, compound, oligo, nucleic acid molecule, or a combination thereof toward modulating the DLX family transcription factor accountable for the biological effect (including the DLX family transcription factor itself). The term “active ingredient” as used herein can also include a genetically modified cell as disclosed herein.

### Pharmaceutically Acceptable Carriers and Excipients

Hereinafter, the phrases “physiologically acceptable carrier” and “pharmaceutically acceptable carrier” are interchangeably used herein to refer to a carrier or a diluent that does not cause significant irritation to an organism and does not abrogate the biological activity and properties of the administered compound. An adjuvant is included under these phrases.

In certain aspects, compositions disclosed herein may further compromise one or more pharmaceutically acceptable diluent(s), excipient(s), and/or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art.

In some aspects, pharmaceutical compositions herein may also include stabilizers, antioxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, or any combination thereof. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In certain aspects, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In some aspects, any of the well-known techniques, carriers, and excipients may be used as suitable and/or as understood in the art.

In certain aspects, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some aspects, pharmaceutical compositions disclosed herein may comprise at least 5%, at least 10%, at least 20%, at least 25%, at least 30%, at least 35%, at least 40%, at least 45%, or at least 50% total amount of polymers as suspending agent(s) by total weight of the composition. In some aspects, pharmaceutical compositions disclosed herein may comprise about 5% to about 99%, about 10%, about 95%, or about 15% to about 90% total amount of polymers as suspending agent(s) by total weight of the composition.

In certain aspects, pharmaceutical compositions disclosed herein may comprise a viscous formulation. In some aspects, viscosity of composition herein may be increased by the addition of one or more gelling or thickening agents. In some aspects, compositions disclosed herein may comprise one or more gelling or thickening agents in an amount to provide a sufficiently viscous formulation to remain on treated tissue. In some aspects, pharmaceutical compositions disclosed herein may comprise at least 5%, at least 10%, at least 20%, at least 25%, at least 30%, at least 35%, at least 40%, at least 45%, or at least 50% total amount of gelling or thickening agent(s) by total weight of the composition. In some aspects, pharmaceutical compositions disclosed herein may comprise about 5% to about 99%, about 10%, about 95%, or about 15% to about 90% total amount of gelling or thickening agent(s) by total weight of the composition. In some aspects, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In certain aspects, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, or at least 50% total amount of one or more agents by total weight of the composition. In some aspects, pharmaceutical compositions disclosed herein may comprise about 5% to about 99%, about 10%, about 95%, or about 15% to about 90% total amount of one or more agents by total weight of the composition. In some aspects, one or more of these agents may be added to improve the performance, efficacy, safety, shelf-life and/or other property of the muscarinic antagonist composition of the present disclosure. In some aspects, additives may be biocompatible, without being harsh, abrasive, and/or allergenic.

In certain aspects, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In some aspects, pharmaceutical compositions disclosed herein may comprise at least 5%, at least 10%, at least 20%, at least 25%, at least 30%, at least 35%, at least 40%, at least 45%, at least 50% total amount of one or more acidifying agents by total weight of the composition. In some aspects, pharmaceutical compositions disclosed herein may comprise about 5% to about 99%, about 10%, about 95%, or about 15% to about 90% total amount of one or more acidifying agents by total weight of the composition.

In certain aspects, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more alkalizing agents by total weight of the composition. In some aspects, pharmaceutical compositions disclosed herein may comprise about 5% to about 99%, about 10%, about 95%, or about 15% to about 90% total amount of one or more alkalizing agents by total weight of the composition.

In certain aspects, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite and other materials known to one of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more antioxidants by total weight of the composition. In some aspects, pharmaceutical compositions disclosed herein may comprise about 5% to about 99%, about 10%, about 95%, or about 15% to about 90% total amount of one or more antioxidants by total weight of the composition.

In certain aspects, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more buffering agents by total weight of the composition. In some aspects, pharmaceutical compositions disclosed herein may comprise about 5% to about 99%, about 10%, about 95%, or about 15% to about 90% total amount of one or more buffering agents by total weight of the composition.

In some aspects, the amount of one or more buffering agents may depend on the desired pH level of a composition. In some aspects, pharmaceutical compositions disclosed herein may have a pH of about 6 to about 9. In some aspects, pharmaceutical compositions disclosed herein may have a pH greater than about 8, greater than about 7.5, greater than about 7, greater than about 6.5, or greater than about 6.

In certain aspects, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition. In some aspects, pharmaceutical compositions disclosed herein may comprise about 5% to about 99%, about 10%, about 95%, or about 15% to about 90% total amount of one or more preservatives by total weight of the composition.

In certain aspects, pharmaceutical compositions disclosed herein may comprise one or more surface-acting reagents or detergents. In some aspects, surface-acting reagents or detergents may be synthetic, natural, or semi-synthetic. In some aspects, compositions disclosed herein may comprise anionic detergents, cationic detergents, zwitterionic detergents, ampholytic detergents, amphoteric detergents, nonionic detergents having a steroid skeleton, or a combination thereof. In some aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition. In some aspects, pharmaceutical compositions disclosed herein may comprise about 5% to about 99%, about 10%, about 95%, or about 15% to about 90% total amount of one or more surface-acting reagents or detergents by total weight of the composition.

In certain aspects, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition. In some aspects, pharmaceutical compositions disclosed herein may comprise about 5% to about 99%, about 10%, about 95%, or about 15% to about 90% total amount of one or more stabilizers by total weight of the composition.

In some aspects, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some aspects, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some aspects, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition. In some aspects, pharmaceutical compositions disclosed herein may comprise about 5% to about 99%, about 10%, about 95%, or about 15% to about 90% total amount of one or more tonicity modifiers by total weight of the composition.

### Dosage Formulations

In certain aspects, the present disclosure provides compositions formulated for one or more routes of administration. Suitable routes of administration may, for example, include intravenous, intracranial, intrathecal, subcutaneous, intranasal route cranial, transmucosal, trans-nasal, transcranial, intracerebroventricular, intestinal, and/or parenteral delivery. In some aspects, compositions herein formulated can be formulated for parenteral delivery. In some aspects, compositions herein formulated can be formulated intramuscular, subcutaneous, intramedullary, intravenous, intraperitoneal, intracranial and/or intranasal injections.

In certain aspects, one may administer a composition herein in a local or systemic manner, for example, via local injection of the pharmaceutical composition directly into a tissue region of a patient. In some aspects, a pharmaceutical composition disclosed herein can be administered parenterally, e.g., by intravenous injection, intracerebroventricular injection, intra-cisterna magna injection, intra-parenchymal injection, or a combination thereof. In some aspects, a pharmaceutical composition disclosed herein can administered to subject as disclosed herein. In some aspects, a pharmaceutical composition disclosed herein can administered to human patient. In some aspects, a pharmaceutical composition disclosed herein can administered to a human patient via two or more administration routes. In some aspects, the combination of administration routes by be intracerebroventricular injection and intravenous injection; intrathecal injection and intravenous injection; intra-cisterna magna injection and intravenous injection; and/or intra-parenchymal injection and intravenous injection.

In certain aspects, pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

In certain aspects, pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof.

In certain aspects, pharmaceutical compositions described herein may be formulated for parenteral administration, e.g., by bolus injection or continuous infusion. Formulations for injection herein may be presented in unit dosage form, e.g., in ampoules or in multidose containers with optionally, an added preservative. In some aspects, compositions herein may be suspensions, solutions or emulsions in oily or aqueous vehicles, and/or may contain formulatory agents such as suspending, stabilizing and/or dispersing agents.

In certain aspects, pharmaceutical compositions herein formulated for parenteral administration may include aqueous solutions of the active preparation (e.g., a viral vector, a polynucleotide composition, engineered cells or DLX transcriptional factor) in water-soluble form. In some aspects, compositions herein comprising suspensions of the active preparation may be prepared as oily or water-based injection suspensions. Suitable lipophilic solvents and/or vehicles for use herein may include, but are not limited to, fatty oils such as sesame oil, or synthetic fatty acids esters such as ethyl oleate, triglycerides or liposomes. In some aspects, compositions herein comprising aqueous injection suspensions may contain substances which increase the viscosity of the suspension, such as sodium carboxymethyl cellulose, sorbitol, and/or dextran. In some aspects, compositions herein comprising a suspension may also contain one or more suitable stabilizers and/or agents which increase the solubility of the active ingredients (e.g., a viral vector, a polynucleotide composition, engineered cells or DLX transcriptional factor) to allow for the preparation of highly concentrated solutions.

In some aspects, compositions herein may comprise the active ingredient in a powder form for constitution with a suitable vehicle, e.g., sterile, pyrogen-free water-based solution, before use.

Pharmaceutical compositions suitable for use in context of the present disclosure may include compositions wherein the active ingredients can be contained in an amount effective to achieve the intended purpose. In some aspects, a therapeutically effective amount means an amount of active ingredients (e.g., a viral vector, a polynucleotide composition, engineered cells or DLX transcriptional factor disclosed herein) effective to prevent, slow, alleviate or ameliorate symptoms of a disorder (e.g. neural degeneration disorder) or prolong the survival of the subject being treated.

### III. Methods of Use

In some aspects the current disclosure encompasses methods of using the compositions disclosed herein for inducing neural regeneration in a subject in need thereof, using the compositions provided herein. In some aspects the current disclosure stems from the novel discovery that expression of DLX genes in a glial cell results in multilineage reprogramming of said cells to produce neural progenitor cells, which give rise to neurons, astrocytes, and oligodendrocytes. In other words, expression of DLX genes can assist neural regeneration in a subject in need thereof.

In some aspects the subject may be suspected of, predicted to, or diagnosed with a neural injury or a neurodegenerative disease. In some aspects the present disclosure provides methods of treating, attenuating, and preventing a neural injury or neural degenerative disorder, including but not limited to Alzheimer's disease, Parkinson's disease, amyotrophic lateral sclerosis (ALS, also known as Lou Gehrig's disease), multiple sclerosis and multiple system atrophy.

A suitable subject includes a human, a livestock animal, a companion animal, a lab animal, or a zoological animal. In some embodiments, the human includes man, woman, children, elderly, adults, and teens. In some other embodiments, the human is an adult human patient, or a pediatric human patient. In some embodiments, the subject may be a rodent, e.g., a mouse, a rat, a guinea pig, etc. In some embodiments, the subject may be a livestock animal. Non-limiting examples of suitable livestock animals may include pigs, cows, horses, goats, sheep, llamas and alpacas. In some embodiments, the subject may be a companion animal. Non-limiting examples of companion animals may include pets such as dogs, cats, rabbits, and birds. In some embodiments, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In some aspects the disclosure also encompasses use of the compositions disclosed herein to promote multilineage reprogramming of a NG2 glia, fibroblasts, microglia or neural progenitor cells in vitro, in vivo or ex vivo. In some aspects, the method disclosed herein comprises contacting a cell with the compositions disclosed herein. In some aspects the method comprises introducing into the cells provided here a disclosed composition. Methods to introduce gene editing components into a cell in vitro or ex vivo include, but are not limited to, electroporation, sonoporation, use of a gene gun, lipofection, calcium phosphate transfection, use of dendrimers, microinjection, and use of viral vectors. These gene-editing components may comprise one or more of the polynucleotides including DNA, cDNA or RNA, viral vectors, CRISPR, TALEN, zinc finger nuclease (ZFN), meganuclease, Mega-TAL, and transposon-based systems. Viral vector delivery systems can include DNA and RNA viruses, which have either episomal or integrated genomes after delivery to the cell. Examples of viral vectors include, but are not limited to, retroviral vectors, lentiviral vectors, adenovirus vectors, poxvirus vectors; herpesvirus vectors, helper-dependent adenovirus vectors, hybrid adenovirus vectors, Epstein-Bar virus vectors, herpes simplex virus vectors, hemagglutinating virus of Japan (HVJ) vectors, and Moloney murine leukemia virus vectors. In some particular aspects the virus is not an adenovirus associated viral vector.

In some aspects the methods disclosed herein comprise administering to a subject in need thereof, a therapeutically effective amount of the compositions provided herein. Suitable modes of administration are known in the art and further provided herein including but not limited to intravenous, intracranial, intrathecal, subcutaneous, intranasal route, cranial, transmucosal, trans-nasal, transcranial, intracerebroventricular, intestinal, and/or parenteral delivery.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

In some aspects, toxicity and therapeutic efficacy of the active ingredients disclosed herein can be determined by standard pharmaceutical procedures in vitro, in cell cultures or experimental animals. In some aspects, data obtained from these in vitro and cell culture assays and animal studies can be used in formulating a range of dosage for use in a human subject. In some aspects, a dosage for use herein may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1).

In certain aspects, dosage amounts and/or dosing intervals may be adjusted individually to brain or blood levels of the active ingredient that are sufficient to induce or suppress the biological effect (minimal effective concentration, MEC). In some aspects, the MEC for an active ingredient (e.g., DLX transcriptional factor disclosed herein) may vary for each preparation but can be estimated from in vitro data. In some aspects, dosages necessary to achieve the MEC herein may depend on individual characteristics and route of administration. Detection assays can be used to determine plasma concentrations.

In certain aspects, depending on the severity and responsiveness of the condition to be treated, dosing with compositions herein can be of a single or a plurality of administrations, with course of treatment lasting from several days to several weeks or until cure is effected or diminution of the disease state is achieved.

In certain aspects, amounts of a composition herein to be administered will be dependent on the subject being treated, the severity of the affliction, the manner of administration, the judgment of the prescribing physician, and the like. In some aspects, effective doses may be extrapolated from dose-responsive curves derived from in vitro or in vivo test systems.

### IV. Kits

The present disclosure provides kits for use in treating or alleviating a target disease, such as a neural injury or neural degeneration disorder as described herein. In some embodiments, kits herein can include instructions for use in accordance with any of the methods described herein. The included instructions can comprise a description of administration of a composition containing the compositions disclosed herein and optionally the second therapeutic agent, to treat, delay the onset, or alleviate a target disease as those described herein. The kit may further include a description of selecting an individual suitable for treatment based on identifying whether that individual has the target disease, e.g., applying the diagnostic method as described herein. In still other embodiments, the instructions can include a description of administering an antibody to an individual at risk of the target disease.

The instructions relating to the use of a composition disclosed herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., a lymphoproliferative disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). In some embodiments, at least one active agent in the composition can be a polynucleotide, viral vector, host cell comprising a nucleic acid sequence disclosed herein or a DLX transcriptional factor as those described herein.

Kits may optionally provide additional components such as buffers and interpretive information. Normally, the kit includes a container and a label or package insert(s) on or associated with the container. In some embodiments, the present disclosure provides articles of manufacture comprising contents of the kits described above.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1. Study Design for Factors Eliciting Multilineage Reprogramming Of Astrocytes in the Adult Mouse Striatum

During neural development, multipotent neural stem cells (NSCs) sequentially generate neurons and glia that make up the entire central nervous system (1). Postnatally, these NSCs persist only in discrete regions of the adult mammalian brain, namely the neurogenic niches including the subventricular zone (SVZ) of the lateral ventricle and the subgranular zone (SGZ) of the hippocampus (2, 3). Neurons generated from the SVZ-NSCs migrate to the olfactory bulb and play a role in olfaction, whereas those from the SGZ remain in the dentate gyrus and participate in learning and memory (4, 5). In contrast to region-restricted NSCs, the ubiquitously distributed resident glial cells are emerging as a cell source for generation of new neurons through fate reprogramming (6-9). Despite controversies (10-12), fate reprogramming can be accomplished under certain injury paradigms (13-16) or through controlling the expression of a single or a combination of fate-determining factors (17-27). Induction of new neurons from resident glia has been achieved in multiple non-neurogenic regions, such as the striatum, the cortex, the spinal cord, and the retina. Nevertheless, a functional neural network requires not only neurons but also glia. Multilineage differentiation will be ideal to provide all three cell types for neural regeneration. Thus far, such multilineage reprogramming of resident glia remains to be determined. A series of in vivo screens were designed to identify new factors capable of reprogramming the fate of resident striatal astrocytes. It was discovered that a single transcription factor, DLX2, was sufficient to reprogram resident astrocytes to become induced neural progenitor cells (iNPCs). Genetic lineage tracings validated the astrocyte origin and delineated the multiple fates of iNPCs. Single-cell RNA sequencing (scRNA-seq) further revealed that the DLX2-mediated reprogramming process resembles neurogenesis from NSCs.

### Example 2. Experimental Procedure

Animals: Wildtype C57BL/6J mice and the following mutant mice were purchased from the Jackson Laboratory: Aldh1I1-CreERT2 (stock 029655), Pdgfra-CreER™ (stock 018280), Ascl1-CreERT2 (stock 012882), R26R-YFP (stock 006148), R26R-tdT (stock 007914). Both adult male and female mice at 8 weeks of age and older were used for experiments. All mice were housed under a controlled temperature and a 12-h light/dark cycle with free access to water and food in the animal facility. Sample sizes were determined based on prior experiences with immunohistochemical analyses of cell reprogramming. Animal procedures and protocols were approved by the Institutional Animal Care and Use Committee at UT Southwestern.

Tamoxifen and BrdU administration: Tamoxifen (T5648, Sigma) was dissolved at 40 mg/ml in a mixture of sesame oil and ethanol (9:1 by volume) and was intraperitoneally injected at a daily dose of 1 mg/10 g body weight for a period of 3-6 days. BrdU (B5002; Sigma) was dissolved at 0.5 g/L in drinking water and was continuously administered for 4 weeks or the indicated durations.

Virus preparation and intracranial injections: Candidate genes were subcloned through PCR into the third-generation lentiviral vector hGFAP-GFP by replacing GFP at the AgeI and XhoI sites (1, 2). Plasmids were verified through restriction enzyme digestions and DNA sequencing. VSV-G pseudotyped lentiviruses were prepared essentially as previously described (2-4). 3.0 μL of lentivirus (0.5-1×109 pfu/mL) was stereotaxically injected with a Hamilton syringe and a 34-gauge needle into the striatum with the following coordinates: anterior/posterior, +1.0 mm; medial/lateral, +2 mm; and dorsal/ventral from the skull, −3.0 mm. Equal ratios of viruses were mixed for multifactor injections. When applicable, mice were bilaterally injected with different viruses for reduced animal usages.

Immunohistochemistry: Mice were euthanized and transcardially perfused with ice-cold 1×PBS, followed by 4% paraformaldehyde (PFA). Brains were then isolated, post-fixed overnight with 4% PFA at 4° C., and cryoprotected with 30% sucrose solution for at least 24 hr at 4° C. Coronal brain sections were collected at 40-μm thickness with a sliding microtome (Leica) and were stored in anti-freezing solution at −20° C. Immunostainings were conducted essentially as previously described (2). Primary antibodies were listed in Table 1. Alexa Fluor 488-, 555- or 647-conjugated corresponding secondary antibodies from Jackson ImmunoResearch were used for indirect fluorescence. Nuclei were counterstained with Hoechst 33342 (HST). Images were taken with a Zeiss LSM700 confocal microscope. For quantifying, 3 of 20× images were taken for each slice, with 3 slices per animal. (Data were obtained from one-sixth of the sections spanning the virus-injected striatal region in each mouse.) Animals with failed or mistargeted viral injections were excluded. A representative image was shown from at least three similar images.

TABLE 3
Information on Primary Antibodies

| Primary | | Cat | | dilution |
|---|---|---|---|---|
| antibody | host | number | Sources | ratio |
| DCX | rabbit | sc-8066, | Cell signaling | 1:500 |
| NEUN | rabbit | Ab 177487 | Abcam | 1:1000 |
| BrdU | rat | OBT0030 | Accurate Chemical | 1:300 |
| CHAT | goat | Ab144P | Fisher(millipore) | 1:500 |
| GABA | rabbit | A2052 | Sigma | 1:1000 |
| APC | mouse | OP80 | Millipore | 1:500 (2 d) |
| ALDH1L1 | mouse | 75-140 | Neuromab | 1:50 (F) |
| OLIG2 | rabbit | Ab9610 | Millipore | 1:1000 |
| Parvalbumin | mouse | P3088 | Sigma | 1:1000 |
| Somatostatin | rabbit | PA2054 | Boster | 1:500 (2 d) |
| DARPP32 | rabbit | Ab40801 | Abcam | 1:500 |
| CTIP2 | rat | Ab18465 | Abcam | 1:500 |
| Calretinin | rabbit | Ab702 | Novus | 1:100 |
| Calbindin | mouse | C9848 | Sigma | 1:100 (F) |
| NG2 | rabbit | Ab5320 | Millipore | 1:250 |
| GFP | chicken | GFP-1020 | Ayes | 1:500 |
| IBA1 | rabbit | 019-19741 | Wako chemicals | 1:200 |
| VGLUT2 | mouse | #135421 | Synaptic System | 1:500 |
| VGAT | mouse | #130011 | Synaptic System | 1:500 |
| ASCL1 | guinea | TX518 | Kim et al. (2008) | 1:1,000 |
| | pig | | | |
| TH | chicken | TH | Ayes | 1:500 |
| DLX2 | rabbit | Ab5726 | Millipore | 1:500 |

Single cell isolation: Adult Aldh1I1-CreERT2; Rosa-tdT mice were treated daily with tamoxifen for 3 days. 7 days later, they were bilaterally injected into the striatum with lentivirus hGFAP-GFP (n=4) or hGFAP-DLX2-IRES-GFP (n=5). At 4 wpv, mice were euthanized with isoflurane and perfused with cold 0.9% NaCl solution to remove blood. Brains were collected and dissected in HABG containing Hibernate A (BrainBits), B27 (Gibco) and Glutamine (Gibco) on ice. Tissues were chopped into ˜0.5-1 mm3 pieces and digested at 37° C. for 45 min in Hibernate A minus Ca medium containing 1 mg/mL papain, 0.125× Glutamine, 0.5 mM EDTA, 0.05 mg/mL DNaseI, and 2.5 mM MgCl2. Digestion was terminated by adding an equal volume of 2% BSA-containing HABG. Cells were dispensed through trituration by using a 10-mL serological pipette for 12 times and were left to stay for 2 min. The supernatant was transferred into a new 50 mL tube. Volume was topped to 1 mL with 1% BSA-containing HABG. Cells were triturated again for 10 times by using a 1-mL low binding barrier tip (Genesee Scientific, #23-430). Cells were combined, filtered through a 40-μm cell strainer (FALCON, REF352340), pelleted down through centrifugation at 200 rcf, and resuspended in 1% BSA-containing HABG. tdT+ cells were sorted into 1% BSA-containing HABG, pelleted and resuspended in 0.05% BSA-containing DPBS for 10× Genomics library construction

10× Genomics single cell RNA sequencing procedure: The concentration of single cell suspension was adjusted to 900-1,000 cells/μL and cells were loaded on the 10× Genomics Chromium system (10× Genomics, Pleasanton, CA) with the aim of generating 10,000 transcriptomes per channel (Chromium Next GEM Single Cell 3′ GEM, Library & Gel Bead Kit v3.1 PN-1000121; Chromium Next GEM Chip G Single Cell Kit, 48 rxns PN-1000120). Single cell RNA sequencing libraries were constructed following the manufacturer's instructions.

Sequencing, basecalling and demultiplexing: Libraries were sequenced on Illumina NextSeq 500/550 sequencing systems (Illumina, San Diego, CA). The mkfastq function from Cell Ranger pipeline (version 3.0.2) was used to perform basecalling and demultiplexing for downstream analyses.

Read alignment and generation of gene expression matrix: For 10× Genomics data, the gene expression matrix was generated for each experiment using the Cell Ranger pipeline (version 3.1.0) with default parameters, except the parameter of expected number of cells, which was adjusted based on each individual experiment. A custom genome was used for mapping, which was based on mm10 with two additional chromosomes representing the lentiviral construct and the Rosa-tdTomato lineage tracing locus. The gene annotation used is based on Ensembl release 84 (GENCODE Gene Set M9).

### Bioinformatic Analysis of Lenti-DLX2 and Lenti-GFP Cells:

Doublet removal: Doublets were removed by Python package Scrublet (version 0.2.3) (Wolock et al., Cell Systems, 2019) for each sample separately, with default settings.

Filtering low quality cells and uninformative genes. Cells with fewer than 500 or greater than 40,000 UMIs were removed. Cells with >20% UMIs derived from mitochondrial genes were removed. Cells with fewer than 400 genes detected were removed. Genes detected in fewer than 5 cells were filtered. 5,756 and 4,610 cells from Lenti-DLX2 and Lenti-GFP samples remained after filtering, respectively.

Calculating highly variable genes, normalizing, scaling, and centering data. The single-cell gene expression matrix was natural log-transformed. Then, the top 2,500 highly variable genes were calculated using the function ‘pp.recipe_zheng17’ from Python package Scanpy (version 1.4.5.post1) (5). Option ‘log=False’ was used since the matrix was already log-transformed. The function also subsets the matrix with the 2,500 highly variable genes, normalizes the total UMI count per cell, scales each gene to unit variance and shifts the mean of each gene across all cells to zero. Principal component analysis (PCA). The resulting matrix was used for PCA with function ‘tl.pca’ from Python package Scanpy (version 1.4.5.post1). Option ‘n_comps=50’ was used to calculate the first 50 principal components.

Batch correction. The first 50 principal components were used as input to the batch correction method Harmony (6). A Python implementation of Harmony, harmonypy, was used (version 0.0.4, https://github.com/slowkow/harmonypy). Batch correction between libraries was used. Function ‘run_harmony’ ran until convergence. The corrected 50 principal components were used to replace the original principal components for downstream analyses.

UMAP visualization. To visualize different cell types in the transcriptional space, unsupervised clustering, and embedded the clusters in a two-dimensional space was performed. Functions ‘pp.neighbors’, ‘tl.louvain’, ‘tl.paga’ from Python package Scanpy (version 1.4.5.post1) were used with default settings. For ‘pp.neighbors’, parameter ‘use_rep=‘Harmony’’ was used to take advantage of the corrected principal components. For ‘tl.louvain’, parameter ‘resolution=0.5’ was used for identification of broad cell clusters. Lastly, function ‘tl.umap’ was used to create single-cell embedding of the scRNA-seq data, with parameter ‘init_pos=‘paga’’ to take advantage of the partition-based graph abstraction embedding of the cell clusters.

### Analysis of the Main Trajectory of Lenti-DLX2 and Lenti-GFP Cells:

Selection of cells. Neural cell clusters with enrichment for cells from the DLX2-induced group of mice were selected for further analyses (i.e., astrocytes, Lenti-astrocytes, NPCs, and neuroblasts). Cell IDs were used to subset the full dataset above, including the gene expression matrix and the 50 corrected principal components by Harmony. Neighbors, clusters, partition-based graph abstraction embedding, and UMAP visualization were re-calculated based on the subset corrected principal components, in the same way as described above. For ‘tl.louvain’, parameter ‘resolution=1.0’ was used for a high-resolution identification of cell clusters.

Pseudotime analysis. A cell in the cluster ‘Astrocytes-1’ was selected as the root of the pseudotime analysis (i.e. start of pseudotime), as reprogramming starts with resident astrocytes in the brain. Function ‘tl.dpt’ from the Python package Scanpy (version 1.4.5.post1) was used to calculate pseudotime with default setting. Last, ranks of pseudotime were calculated for all cells, with low rank (rank=0) corresponding to the beginning of pseudotime, and high rank to the end.

Gene clustering analysis. To identify genes underlying reprogramming, cells were excluded from the control group from further analyses. To identify genes with non-uniform expression across clusters, a Chi-square test with the null hypothesis being uniform expression of a gene across all clusters was performed. This analysis identified 2,584 genes with a multiple testing-adjusted p value<1e-100. Next, the UMI count matrix of these genes was normalized, scaled, and centered using the function ‘pp.recipe_zheng17’ from the Python package Scanpy (version 1.4.5.post1). Lastly, the neighbors of each gene were identified and unsupervised clustering using the Scanpy functions ‘pp.neighbors’ and ‘tl.louvain’ was performed.

Gene ontology enrichment analysis. For each cluster of genes, gene ontology enrichment analysis was performed with the function ‘GOEnrichmentStudyNS’ from the Python package goatools (version 0.9.9), like stated above.

Gene regulatory network (regulons) reconstruction. Among the 4,607 lenti-DLX2 group cells in the main trajectory, pySCENIC (7) (version 0.10.0) was used to reconstruct regulons. Default settings were used for all functions. The top 10 specific regulons for each cell cluster are shown in the heat map.

Enrichment of putative Dlx1/2 targets. Genes down-regulated in Dlx1/2 knockout in published paper (8) were overlapped with cluster 4 genes or genes in Dlx1 regulon. Genes associated with chromatin activation by Dlx1/2 in published paper (8) were overlapped with genes in Dix1 regulon. Chi-square test was used to determine the p value.

Aggregation of experimental and public WT E18.5 brain datasets: The aggr function from Cell Ranger pipeline (version 3.1.0) was used to generate a combined gene expression matrix from the gene expression matrices of individual experiments. Option ‘--normalize=mapped’ was used to normalize the different sequencing depths between experiments.

### Bioinformatic Co-Analysis of Reprogrammed and WT E18.5 Cells:

Public WT E18.5 brain data. BAM files of two libraries (two BAM files per library) from the dataset ‘1.3 Million Brain Cells from E18 Mice’ from 10× Genomics were downloaded (GEO accession: SRR5167890, SRR5259345, SRR5167928, SRR5259383). BAM files are converted to Fastq files by using the software ‘bamtofastq’ (version 1.2.0) from 10× Genomics. Cells are from cortex, hippocampus and subventricular zone of two E18 C57BL/6 mice.

Filtering low quality cells and uninformative genes. After Lenti-DLX2, Lenti-GFP, and WT E18.5 cells were combined, cells with fewer than 500 or greater than 40,000 UMIs were removed. Cells with >20% UMIs derived from mitochondrial genes were removed. Cells with fewer than 400 genes detected were removed. Genes detected in fewer than 10 cells were filtered. 5,756, 4, 611, and 19,964, cells remained after filtering from Lenti-DLX2, Lenti-GFP, and WT E18.5, respectively.

Calculating highly variable genes, normalizing, scaling, and centering data. The single-cell gene expression matrix was natural log-transformed. Then, the top 2,500 highly variable genes were calculated using the function ‘pp.recipe_zheng17’ from Python package Scanpy (version 1.4.5.post1) (5). Option ‘log=False’ was used since the matrix was already log-transformed. The function also subsets the matrix with the 2,500 highly variable genes, normalizes the total UMI count per cell, scales each gene to unit variance and shifts the mean of each gene across all cells to zero.

Principal component analysis (PCA). The resulting matrix was used for PCA with function ‘tl.pca’ from Python package Scanpy (version 1.4.5.post1). Option ‘n_comps=50’ was used to calculate the first 50 principal components.

Batch correction. The first 50 principal components were used as input to the batch correction method Harmony (6). A Python implementation of Harmony, harmonypy, was used (version 0.0.4, https://github.com/slowkow/harmonypy). Multivariate correction first between batches (reprogramming vs public WT E18.5), and then between libraries, was used. Function ‘run_harmony’ ran until convergence. The corrected 50 principal components were used to replace the original principal components for downstream analyses.

UMAP visualization. To visualize different cell types in the transcriptional space, unsupervised clustering, and embedded the clusters in a two-dimensional space was performed. Functions ‘pp.neighbors’, ‘tl.louvain’, ‘tl.paga’ from Python package Scanpy (version 1.4.5.post1) were used with default settings. For ‘pp.neighbors’, parameter ‘use_rep=‘Harmony’’ was used to take advantage of the corrected principal components. For ‘tl.louvain’, parameter ‘resolution=0.5’ was used for identification of broad cell clusters. Lastly, function ‘tl.umap’ was used to create single-cell embedding of the scRNA-seq data, with parameter ‘init_pos=‘paga’’ to take advantage of the partition-based graph abstraction embedding of the cell clusters.

### Analysis of the Main Trajectory of Reprogrammed and WT E18.5 Cells:

Selection of cell clusters. The full dataset was subsetted like stated above to focus on neural clusters with enrichment of Lenti-DLX2 cells over Lenti-GFP. Likewise, neighbors, clusters, partition-based graph abstraction embedding, and UMAP visualization were re-calculated.

Pseudotime analysis. A cell in the cluster ‘resident astrocytes’ was selected as the root of the pseudotime analysis (i.e. start of pseudotime), The remaining analysis was performed as indicated above.

Differential expression analysis. For each stage on the main trajectory, differential expression analysis was performed between 70 randomly selected experimental and 70 randomly selected WT E18.5 cells. This was repeated for a total of 100 times to measure the variation of the number of differentially expressed genes. Function ‘api.test.two_samples’ with option ‘test=‘rank’’ from the Python package diffxpy was used (https://github.com/theislab/diffxpy/) (version 0.7.4+16.g3689ea8) to perform differential expression analysis.

Gene ontology enrichment analysis. For each set of differentially expressed genes (reprogramming enriched or WT E18.5 enriched), function ‘GOEnrichmentStudyNS’ from the Python package goatools (version 0.9.9) (9) was used to perform gene ontology enrichment analysis. All mouse protein-coding genes were set as background. Only enriched gene ontology terms with multiple testing-adjusted p value<0.05 were considered as statistically significantly enriched.

### Bioinformatic Co-Analysis of Reprogrammed and Adult Hippocampal Neurogenesis Cells:

Public adult hippocampal data. Expression matrix of Dataset C in Hochgerner et al., Nature Neuroscience, 2018 was downloaded (GSE104323). The original matrix contains 24,185 cells and 27,933 genes.

Filtering low quality cells and uninformative genes. Adult hippocampal cells with >20% UMIs derived from mitochondrial genes were removed. No filtering based on UMIs or numbers of genes detected was done because the data was already reasonably good with these regards. Genes detected in fewer than 10 cells were filtered. 23,831 cells remained after filtering. Afterwards, this expression matrix was combined with the combined filtered expression matrix of Lenti-DLX2 and Lenti-GFP cells, with 16,246 genes in common between the two matrices kept.

Calculating highly variable genes, normalizing, scaling, and centering data. The single-cell gene expression matrix was natural log-transformed. Then, the top 2,500 highly variable genes were calculated using the function ‘pp.recipe_zheng17’ from Python package Scanpy (version 1.4.5.post1) (5). Option ‘log=False’ was used since the matrix was already log-transformed. The function also subsets the matrix with the 2,500 highly variable genes, normalizes the total UMI count per cell, scales each gene to unit variance and shifts the mean of each gene across all cells to zero.

Principal component analysis (PCA). The resulting matrix was used for PCA with function ‘tl.pca’ from Python package Scanpy (version 1.4.5.post1). Option ‘n_comps=50’ was used to calculate the first 50 principal components.

Batch correction. The first 50 principal components were used as input to the batch correction method Harmony (6). A Python implementation of Harmony, harmonypy, was used (version 0.0.4, https://github.com/slowkow/harmonypy). Multivariate correction first between batches (reprogramming vs adult neurogenesis), and then between libraries, was used. Function ‘run_harmony’ ran until convergence. The corrected 50 principal components were used to replace the original principal components for downstream analyses.

UMAP visualization. To visualize different cell types in the transcriptional space, unsupervised clustering, and embedded the clusters in a two-dimensional space was performed. Functions ‘pp.neighbors’, ‘tl.louvain’, ‘tl.paga’ from Python package Scanpy (version 1.4.5.post1) were used with default settings. For ‘pp.neighbors’, parameter ‘use_rep=‘Harmony’’ was used to take advantage of the corrected principal components. For ‘tl.louvain’, parameter ‘resolution=0.5’ was used for identification of broad cell clusters. Lastly, function ‘tl.umap’ was used to create single-cell embedding of the scRNA-seq data, with parameter ‘init_pos=‘paga’’ to take advantage of the partition-based graph abstraction embedding of the cell clusters.

### Analysis of the Main Trajectory of Reprogrammed and Adult Hippocampal Neurogenesis Cells:

Selection of cell clusters. The full dataset was subsetted like stated above to focus on neural clusters with enrichment of Lenti-DLX2 cells over Lenti-GFP. Likewise, neighbors, clusters, partition-based graph abstraction embedding, and UMAP visualization were re-calculated.

Pseudotime analysis. A cell in the cluster ‘Astrocytes-2’ was selected as the root of the pseudotime analysis (i.e. start of pseudotime), The remaining analysis was performed as indicated above.

Statistical analysis: Quantification data were presented as mean #SEM. Statistical analysis was performed by homoscedastic two-tailed Student's t-test or one-way ANOVA using the GraphPad Prism software v. 9.3. A p value<0.05 was considered significant. Significant differences are indicated by *p<0.05, **p<0.01, ***p<0.001, and ****p<0.0001.

The single-cell RNA-seq datasets generated in this disclosure can be accessed through https://www.ncbi.nlm.nih.gov/geo/query/acc.cgi?acc=GSE154213. Code for scRNA-seq analyses can be accessed through https://github.com/liboxun/A-single-factor-elicits-multilineage-reprogramming-of-astrocytes-in-the-adult-mouse-striatum. All sequences were incorporated by reference.

### Example 3. Experimental Results

### DLX2 Efficiently Initiates Neurogenesis in the Adult Mouse Striatum

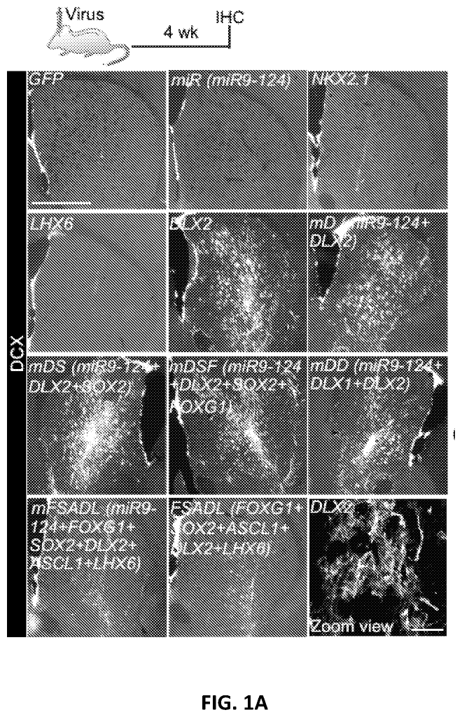

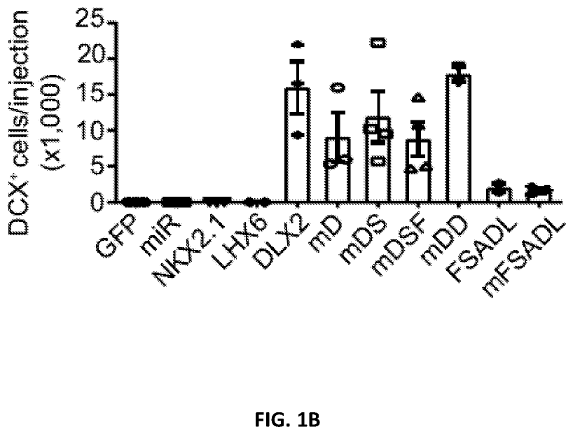

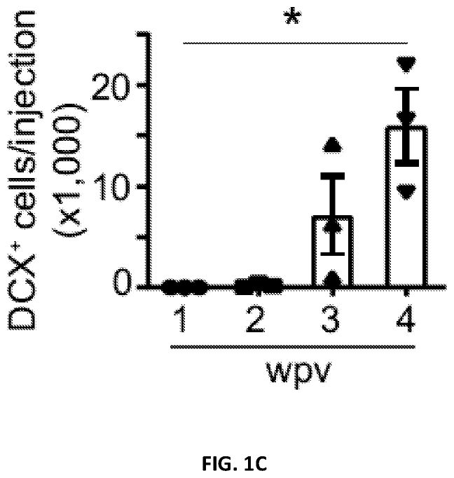

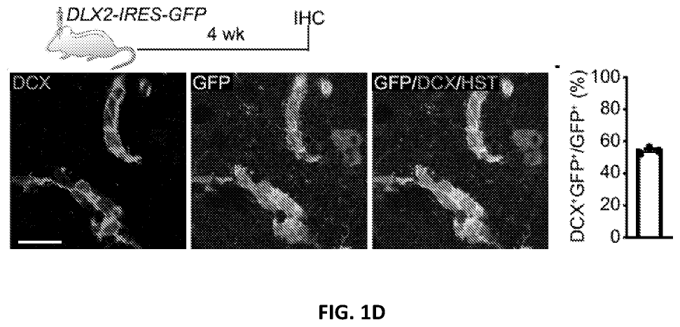

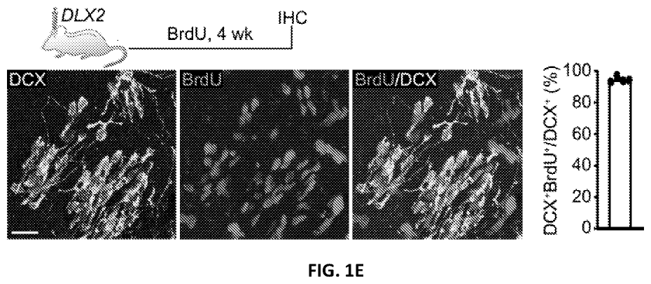

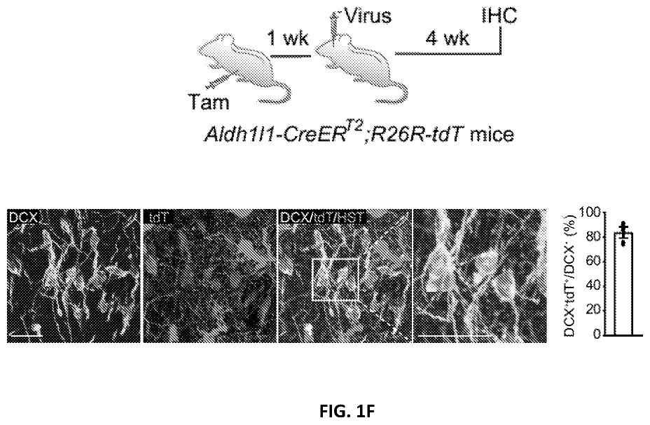

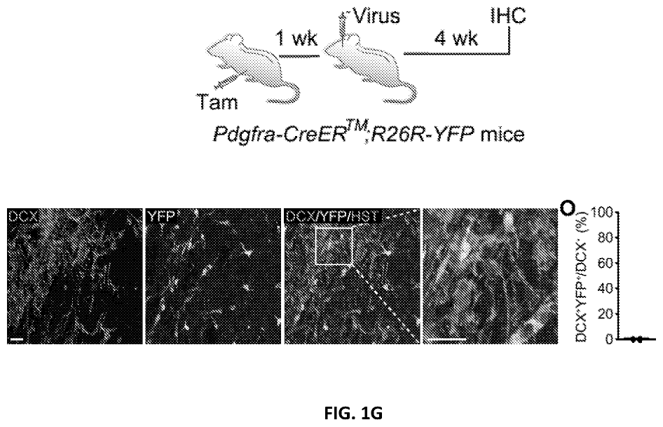

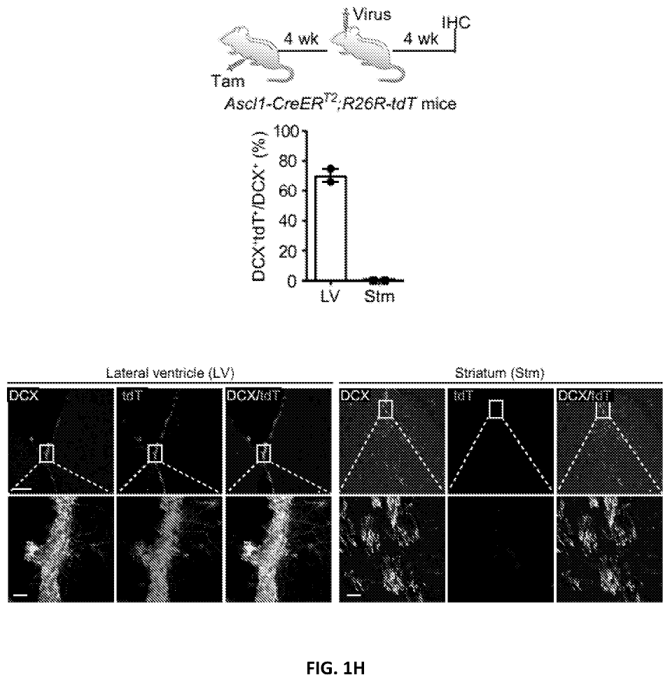

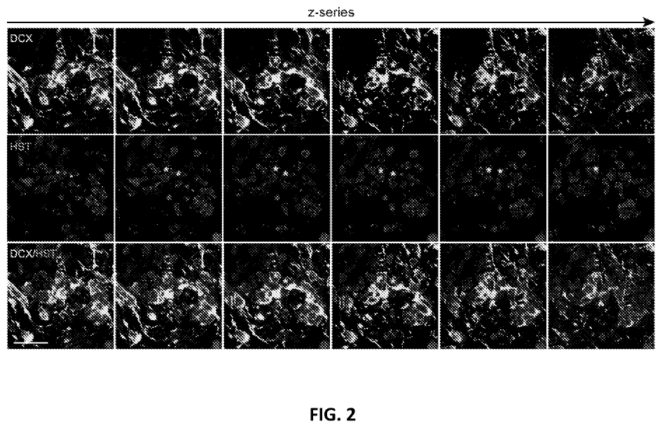

A series of in vivo screens were conducted for identifying factors that could induce neurogenesis in the adult mouse striatum. Transcription factors specifying GABAergic neurons, which are the predominant subtype in the striatum, were tested. These factors were delivered into the striatum through lentivirus under the hGFAP promoter, which mainly targets astrocytes (18). New neurons were initially analyzed by staining for DCX, a marker that is transiently expressed in neuroblasts and immature neurons but absent in the adult striatum (17, 18). These cells were imaged and quantified with a confocal microscope (FIG. 2). When examined at 4 weeks post virus-injections (wpv), DCX⁺ cells were not detectable in the striatum injected with virus expressing miR9-124, NKX2.1 or LHX6 (FIGS. 1A, 1B). In contrast, the DLX2-containing groups all produced DCX⁺ cells. A time-course analysis of the DLX2 alone group showed that DCX⁺ cells were not detectable at 1 wpv but greatly increased from 2 to 4 wpv (FIG. 1C; p=0.0083 and F (3,8)=8.101 by one-way ANOVA).

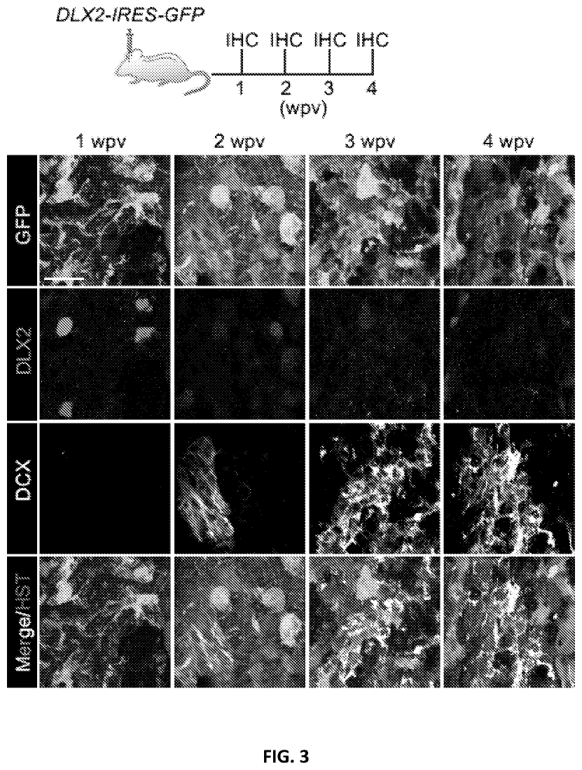

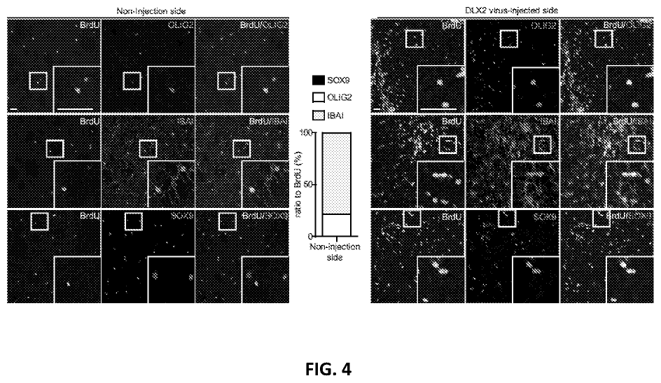

The reprogramming efficiency was estimated by using the co-expressed GFP reporter in a virus expressing DLX2-IRES-GFP (FIG. 1D). Unlike DLX2 that was quickly downregulated after reprogramming, GFP was more stable and could still be detected at 4 wpv (FIG. 3). About half (54.32%) of DLX2 virus-transduced GFP+ cells were transformed into DCX+ cells, suggesting a relatively high efficiency (FIG. 1D). To determine whether these DCX+ cells were newly generated, DLX2-injected mice were treated with BrdU-containing drinking water for 4 weeks (FIG. 1E). In the non-injected contralateral side, BrdU⁺ cells were rarely observed, and they were mainly OLIG2⁺ oligodendrocyte precursor cells (OPCs) or IBA1⁺ microglia (FIG. 4, left panel). In contrast, many BrdU⁺DCX+ cells were detected in the DLX2 virus-injected side (FIG. 1E), with the remaining BrdU⁺DCX-cells being SOX9⁺ astrocytes, OLIG2⁺ OPCs or IBA1⁺ microglia (FIG. 4, right panel). Together, these results indicate that DLX2 efficiently induced DCX⁺ cells, which passed through a proliferative state.

DLX2-Induced Neurogenesis Arises from Resident Astrocytes

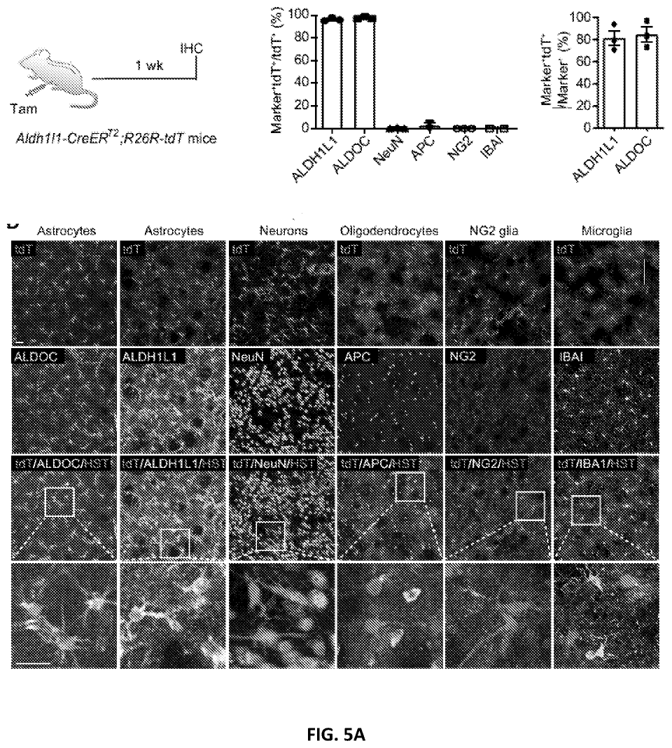

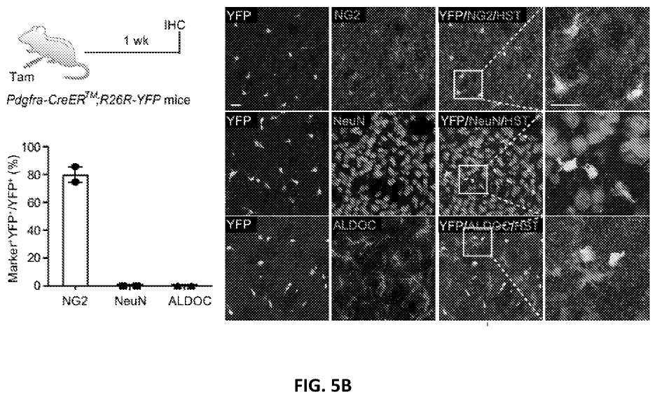

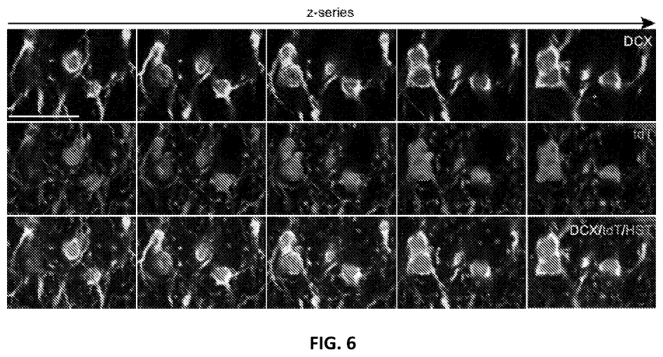

Lineage tracing experiments were conducted to confirm the cell origin for DLX2-induced DCX+ cells. Astrocytes were traced in Aldh1I1-CreERT2; R26R-tdTomato (tdT) mice after 3 daily tamoxifen (Tam) treatments (see schematic in FIG. 5A). 97.18% and 98.39% of tdT⁺ striatal cells were positive for the astrocyte marker ALDH1L1 and ALDOC, respectively (data shown in FIG. 5A). The remaining tdT⁺ cells were APC⁺ oligodendrocytes (2.54±2.54%) and NeuN⁺ neurons (0.21±0.21%). Such results indicate that striatal astrocytes can be efficiently traced in these mice. These mice were then injected with DLX2 virus one week post Tam (FIG. 1F). DCX⁺ and tdT⁺ cells were imaged and quantified with confocal microscopy at 4 wpv (FIG. 6). 83.88% of DLX2-induced DCX⁺ cells were co-labeled with tdT, indicating an origin of resident astrocytes (FIG. 1F).

Since lentivirus under the hGFAP promotor also transduces a fraction of NG2 glia (18), their contribution to DLX2-induced DCX⁺ cells was examined by using the Pdgfra-CreER™; R26R-YFP mice. In the control experiment, 80.16% of NG2 glia but not NeuN⁺ neurons or ALDOC⁺ astrocytes could be traced by the reporter YFP (FIG. 5B), showing cell type-specificity of this lineage tracing mouse line. These mice were then injected with DLX2 virus and examined at 4 wpv (see schematic in FIG. 1G). Immunohistochemistry showed robust induction of DCX⁺ cells in the DLX2-injected striatum; however, none of them were YFP⁺, excluding NG2 glia as a cell origin for these DCX⁺ cells (FIG. 1G).

Previous studies indicated that brain injury may lead to migration of endogenous neuroblasts in the SVZ to the lesion area (29). This raised a possibility that DLX2-induced DCX⁺ cells might just be the migrating endogenous neuroblasts. This possibility was examined in adult Ascl1-CreERT2; R26R-tdT mice (18, 30). These mice were treated with Tam and subsequently injected with virus (FIG. 1H). When examined at 4 wpv, approximately 70.47% of DCX⁺ cells were labeled with tdT in the SVZ, whereas tdT⁺ neuroblasts/progenitors were not observed in the DLX2 virus-injected striatum (FIG. 1H), clearly excluding their contribution to DLX2-induced neurogenesis.

Collectively, these above multiple lineage-tracing results indicate that DLX2-induced DCX+ cells originate from resident astrocytes but not from NG2 glia or SVZ neuroblasts/progenitors.

DLX2 Rapidly Reprograms Astrocytes into ASCL1⁺ INPCs

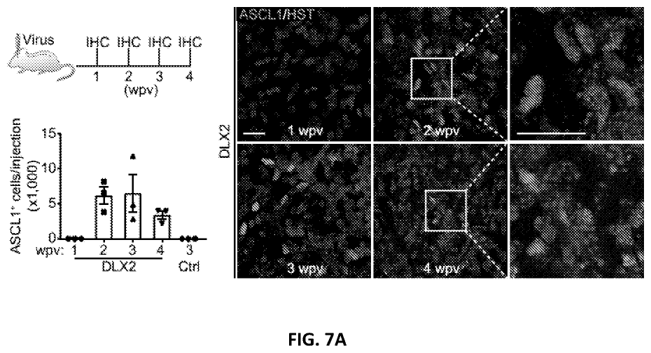

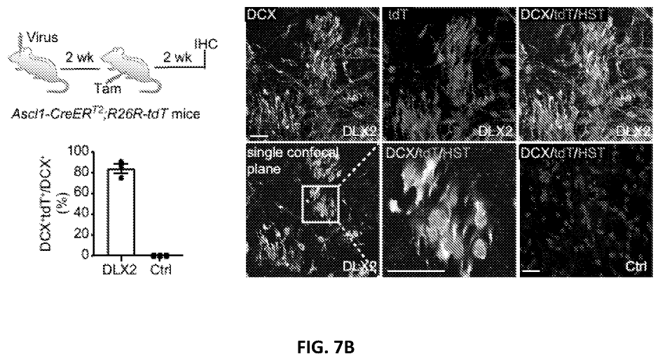

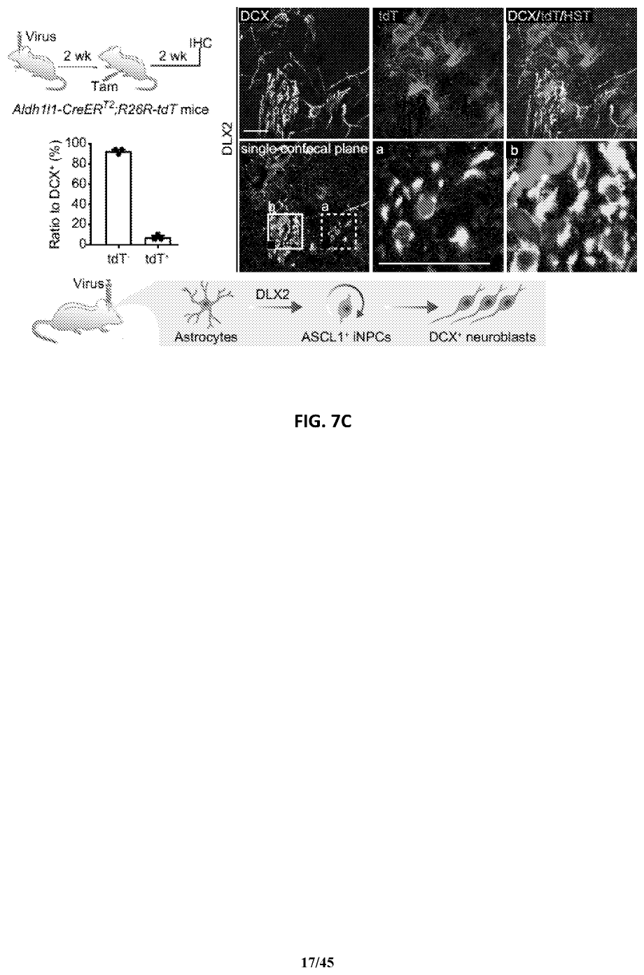

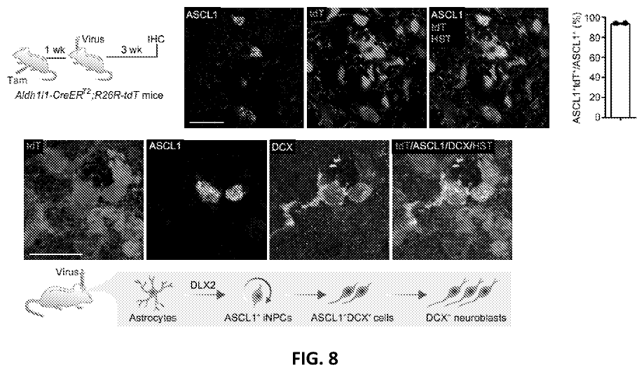

DLX2-mediated reprogramming process was examined by immunohistochemistry and genetic lineage tracing. During endogenous neurogenesis, ASCL1⁺ NPCs preceded DCX⁺ cells (30). While not detected at 1 wpv, DLX2 induced thousands of ASCL1⁺ cells in the injection area at 2 or 3 wpv (FIG. 7A). Genetic lineage tracing in Tam-treated Aldh1I1-CreERT2; R26R-tdT mice confirmed that DLX2-induced ASCL1⁺ cells originated from resident astrocytes (FIG. 8). By 4 wpv, both the expression level and the number of ASCL1⁺ cells were reduced (FIG. 7A). Such a time-course of ASCL1 expression contrasted with that of DCX, which was first robustly detected at 3 wpv and further increased at 4 wpv (FIG. 1C), suggesting that ASCL1⁺ NPCs gave rise to DCX⁺ cells. Indeed, some transitional ASCL1⁺DCX⁺ cells were found at 3 wpv; and these cells were traced back to astrocytes indicating a lineage progression from astrocytes to ASCL1⁺ cells to DCX⁺ neuroblasts (FIG. 8). The adult Ascl1-CreERT2; R26R-tdT mice were employed to further validate such a cell lineage progression. After injection of DLX2 virus, mice were treated with Tam for one week starting at 2 wpv (FIG. 7B). At 4 wpv, about 82.63% of DCX⁺ cells were genetically labeled with the reporter tdT, confirming an origin of the DLX2-induced ASCL1⁺ NPCs (FIG. 7B). Of note, tdT⁺ cells were not observed in the control virus-injected striatum.

Next examined was whether the DLX2-reprogrammed astrocytes lost their astrocyte identity. The adult Aldh1I1-CreERT2; R26R-tdT mice were first injected with DLX2 virus and then treated with Tam starting at 2 wpv for 5 days (see schematic in FIG. 7C). When examined at 4 wpv, it was observed that 92.70% of DCX⁺ cells were not labeled by tdT (data shown in FIG. 7C), indicating that DLX2 has rapidly reprogrammed the fate of resident astrocytes. The remaining 7.3% of DCX⁺tdT⁺ cells were likely derived from those astrocytes still undergoing fate-conversion at 2 wpv. Together, these above results showed that DLX2-reprogrammed resident astrocytes rapidly lose their astrocyte identity and become induced NPCs (INPCs) that further gave rise to DCX⁺ neuroblasts (bottom panel, FIG. 7C).

### INPCs Generate CTIP2⁺ GABAergic Neurons

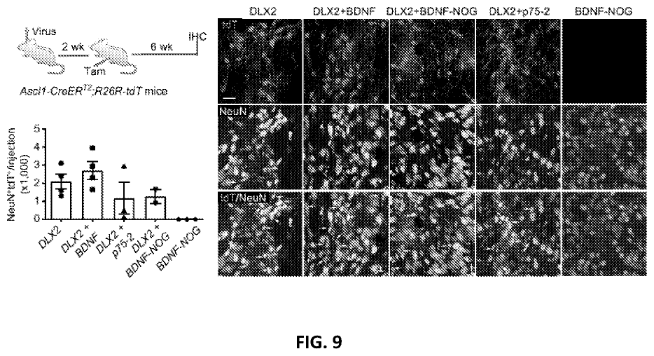

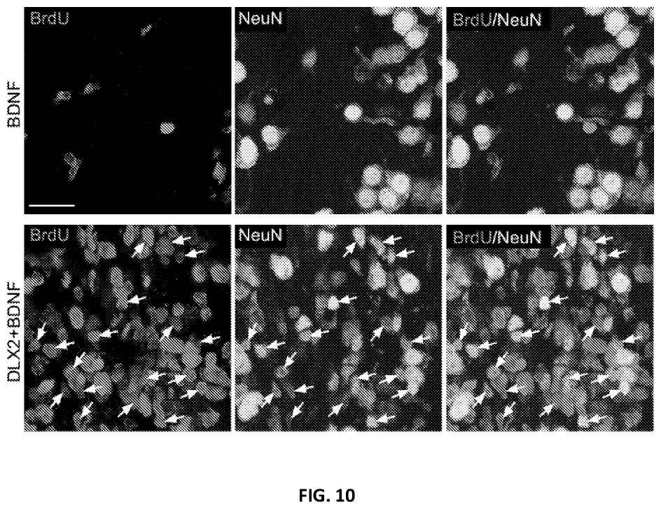

To determine whether iNPCs gave rise to mature neurons, INPCs were traced in Ascl1-CreERT2; R26R-tdT mice after virus injections and Tam treatments (see schematic in FIG. 9). DLX2 alone was able to generate about 2,000 tdT-traced NeuN⁺ neurons when examined at 8 wpv (FIG. 9, bar graph). DLX2 activity was examined to see if it could be promoted by neurotrophic factors including BDNF, NOG, and the NT3 mutant p75-2, all of which were shown to promote neuronal maturation (17, 18, 22, 31). Lentiviruses encoding these factors were co-injected with DLX2 virus and examined at 8 wpv in Ascl1-CreERT2; R26R-tdT mice. BDNF slightly increased whereas BDNF-NOG or p75-2 decreased the number of DLX2-induced NeuN⁺tdT⁺ cells (FIG. 9). Of note, NeuN⁺tdT⁺ cells were not observed in the control BDNF-NOG alone condition. New neurons were confirmed by BrdU-labeling, with abundant BrdU+NeuN+ cells in the DLX2-BDNF group but not in the control BDNF alone group (FIG. 10).

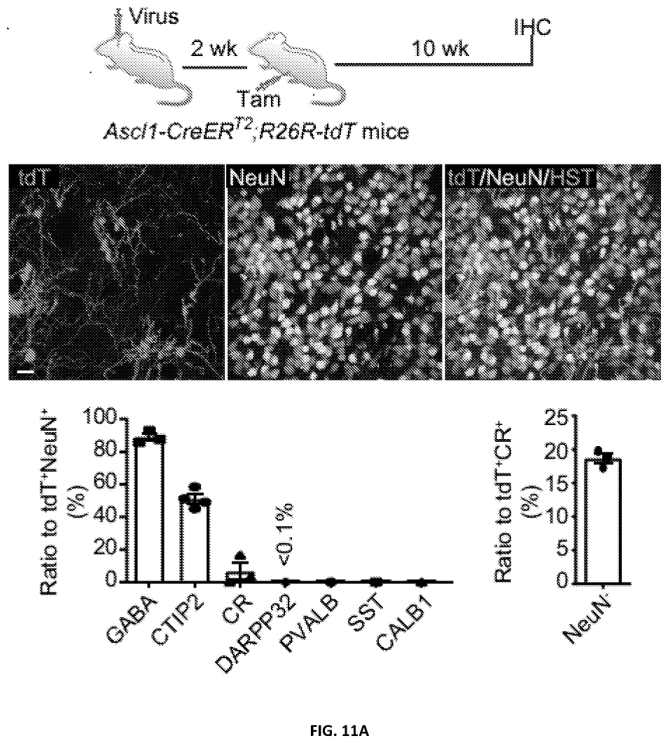

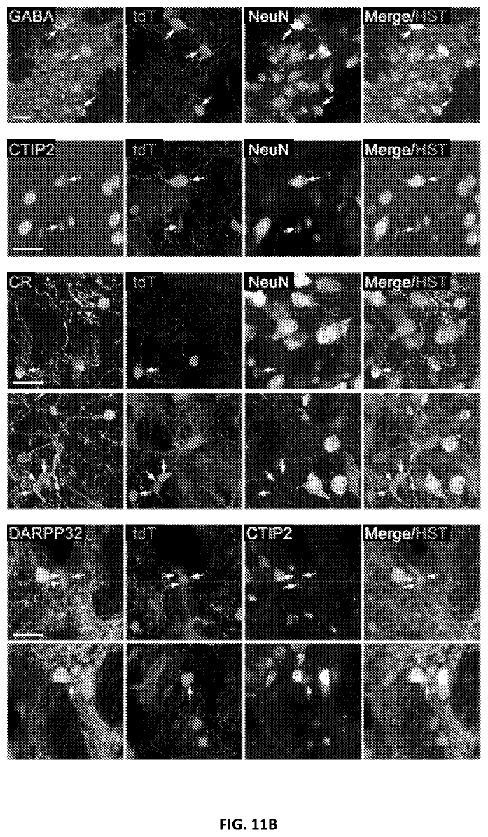

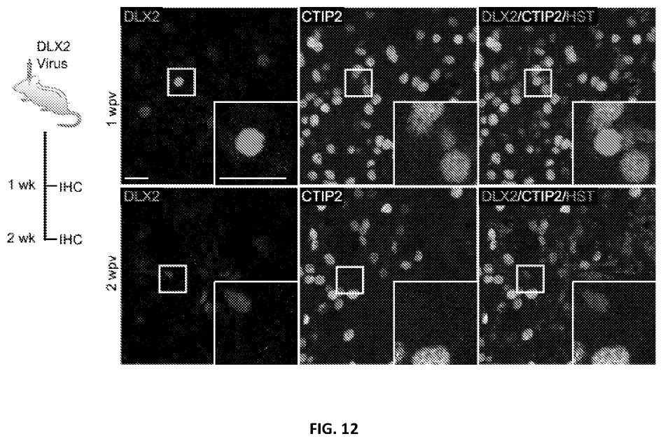

Neuronal subtypes were then examined at 12 wpv in adult Ascl1-CreERT2; R26R-tdT mice (FIG. 11A), a time point at which the induced NeuN⁺tdT⁺ neurons acquired complex morphology (FIG. 11A). Approximately 88.69% of induced neurons were GABAergic, while CHAT⁺ or VGLUT2⁺ neurons were not detected (FIG. 11). Interestingly, CTIP2 (also known as BCL11B), a transcription factor critical for striatal development and specification of medium spiny neurons (32), was observed in 51.00% of NeuN⁺tdT⁺ neurons (FIG. 11A, bar graph bottom right, and FIG. 11B). Since an examination of the early reprogramming process showed that CTIP2 was not directly induced by ectopic DLX2 (FIG. 12), its expression in NeuN⁺tdT⁺NeuN⁺tdT⁺ cells rather suggests that these cells gained a striatal identity. Nonetheless, the number of DARPP32⁺ cells was low, indicating that a majority of these induced CTIP2⁺ neurons were not developed into mature medium spiny neurons at this stage. CALB2 (also known as calretinin) was observed in both NeuNtand NeuN-induced neurons (FIG. 11B, bottom two panels); the latter was also previously detected in the normal mouse forebrain and may represent a new class of calretinin-positive neurons (33). On the other hand, PVALB, SST, or CALB1 was not detected in these NeuN⁺tdT⁺ neurons (FIG. 11A).

iNPCs are Multipotent

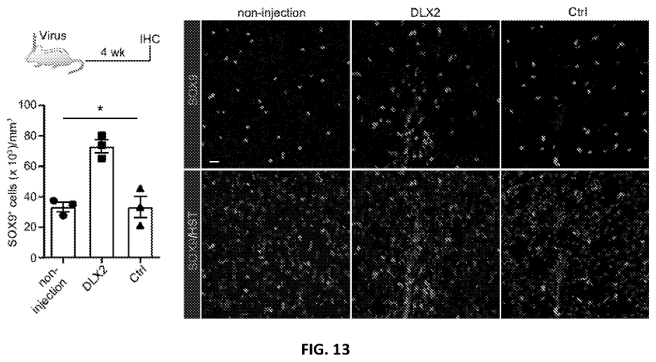

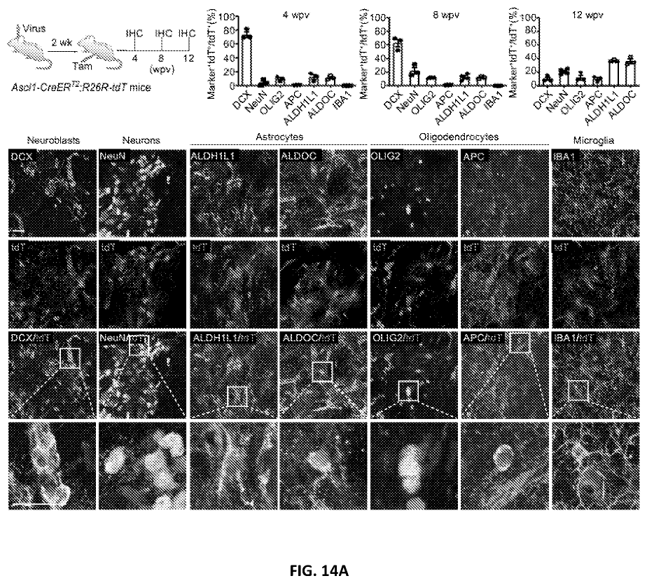

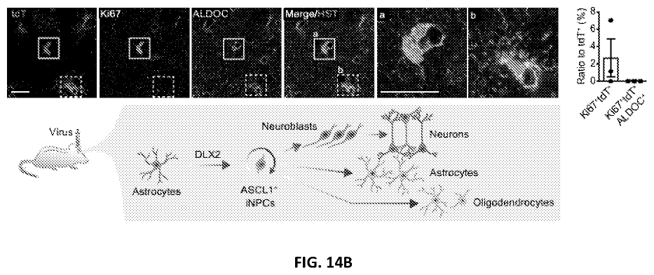

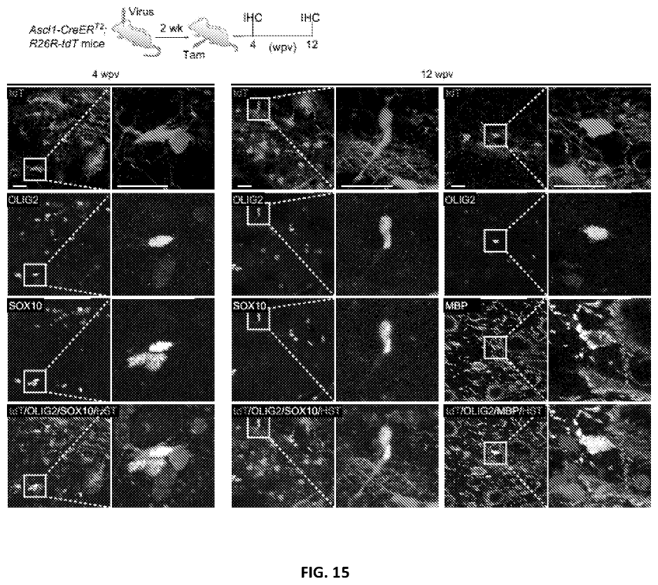

In addition to neuroblasts and neurons, DLX2 induced glia-like cells were also detected in the striatum. An estimate showed a significantly increased number of SOX9⁺ astrocytes surrounding the DLX2-injected regions (FIGS. 13, p=0.0021 and F (2,6)=20.34 by one-way ANOVA). Both neurons and glial cells induced by DLX2 were surveyed through a time-course analysis in adult Ascl1-CreERT2; R26R-tdT mice (FIG. 14A). At 4 wpv, DCX⁺ and NeuN⁺ cells represented about 73.41% and 4.45% of DLX2-induced tdT⁺ cells, respectively (FIG. 14A, bar graph top left). The ratio of glial cells, indicated by OLIG2, ALDH1L1, or ALDOC, was about 12% (FIG. 14A). SOX10⁺OLIG2⁺tdT⁺OPCs were detected at this time point (FIG. 15). By 8 wpv, more neuroblasts differentiated into neurons, leading to a decrease of DCX⁺ cells (62.63%) and a corresponding increase of NeuN⁺ cells (22.00%), whereas the ratios of OLIG2⁺ glia (12%) and ALDH1L1⁺ or ALDOC⁺ astrocytes (12%) remained unchanged (FIG. 14A, bar graph, middle). After another 4 weeks, DCX+ cells decreased to 10.15%, while the ratio of neurons remained constant (FIG. 14A, bar graphs middle and right). In contrast, more APC+tdT⁺ oligodendrocytes appeared, and the fraction of ALDH1L1+tdT⁺ or ALDOC+tdT⁺ astrocytes increased to 36% (FIG. 14A, bar graph, right). Interestingly, both SOX10+OLIG2+tdT⁺OPCs and MBP+OLIG2+tdT⁺ oligodendrocytes could be observed at this later time (FIG. 15). Thus, for the first 8 weeks, the ratio of neurogenesis to gliogenesis was 4:1, with 80% of DLX2-induced tdT⁺ cells as neuroblasts/neurons and the remaining 20% as glial cells. By 12 wpv, the glia fraction greatly increased, with astrocytes and oligodendrocytes representing about half of the total DLX2-induced tdT⁺ cells (FIG. 14A). Two mechanisms might account for the increased number of glial cells at later time points. On one hand, glial cells might proliferate in response to a regeneration niche; however, they morphologically resembled protoplasmic astrocytes (FIG. 14B), and none of them were Ki67+, a marker of proliferating cells (FIG. 14B). On the other hand, INPCs might proliferate and be more prone to differentiate into glial cells. It was observed 2% of all tdT⁺ cells were Ki67⁺ cells (FIG. 14B). They morphologically resembled ASCL1⁺ or OLIG2⁺ cells, both of which might give rise to oligodendrocytes and astrocytes. Thus, INPCs reprogrammed from astrocytes largely sequentially generate neurons and glial cells (FIG. 14B).

scRNA-Seq Captures the DLX2-Mediated Reprogramming Trajectory

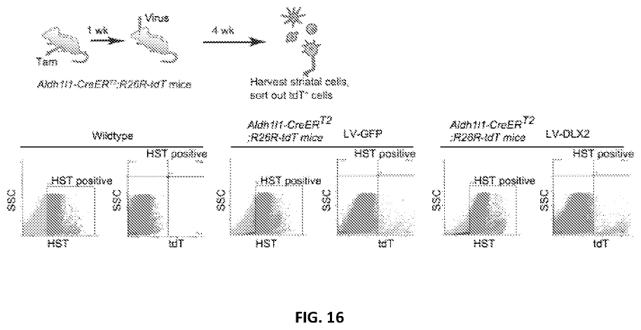

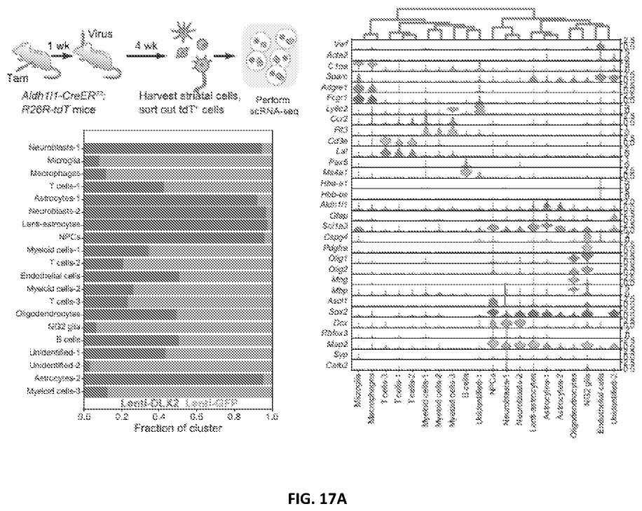

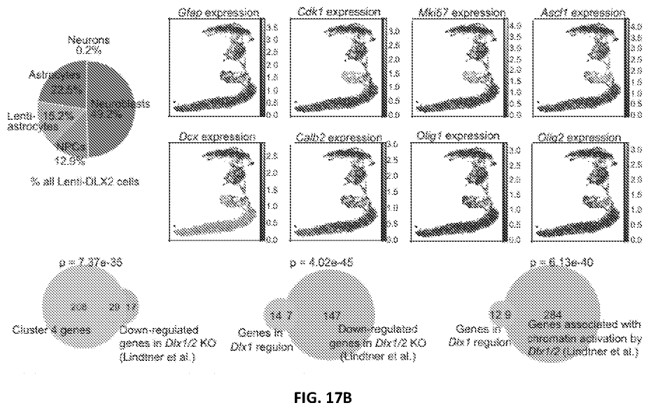

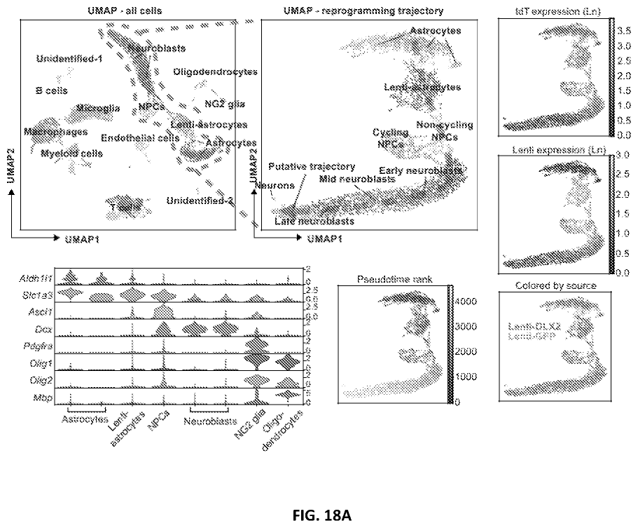

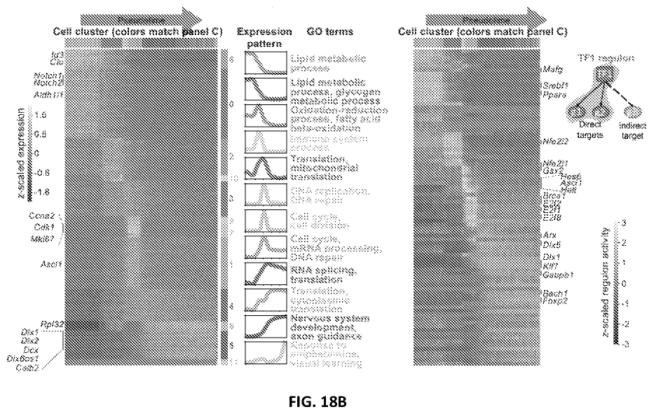

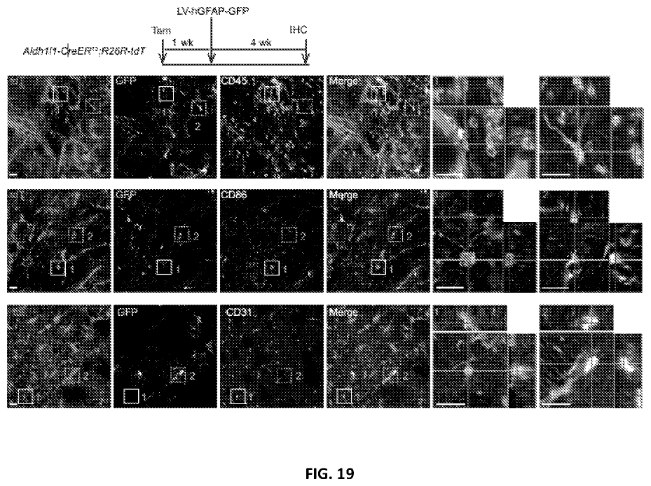

To define the reprogramming process, single-cell RNA sequencing (scRNA-seq) of tdT+ cells was performed, then were sorted from Tam-treated and virus-injected Aldh1I1-CreERT2; R26R-tdT mice at 4 wpv (FIGS. 16, 17A). Two samples were prepared: Lenti-DLX2 (n=5 mice) and Lenti-GFP (n=4 mice). After data quality filtering, this dataset consisted of 5,756 cells from the Lenti-DLX2 group and 4,610 cells from the Lenti-GFP control. Bioinformatic analysis was performed by batch correction (34), clustering, and dimensionality reduction (35)—all standard bioinformatic methods used to analyze scRNA-seq data—to generate a map of all analyzed cells (FIG. 18A). In this map, each cluster of cells represented by the same color suggests that these cells shared transcriptome-wide similarity, and thereby likely represented a distinct cell state, cell type or subtype. A panel of known marker genes was then used to assign broad cell type identity to each cell cluster (FIGS. 18B, 17B). Specifically, expected cell types in the brain were identified, such as neural cells (astrocytes, NPCs, neuroblasts, NG2 glia, and oligodendrocytes), immune cells (T cells, B cells, myeloid cells, macrophages, and microglia), and endothelial cells. Immunohistochemistry confirmed that these non-neural cells could indeed be traced in virus-injected regions of Tam-pretreated Aldh1I1-CreERT2; R26R-tdT mice (FIG. 19).

To focus on cells with the most relevance to DLX2-mediated reprogramming, neural clusters were selected with enrichment for Lenti-DLX2 cells (top middle FIG. 18A, 17A). Among these cells, 22.5% were unperturbed astrocytes, 15.2% were lenti-astrocytes, 12.9% are NPCs, 49.2% were neuroblasts, and 0.2% were neurons (FIG. 17B). Pseudotime analysis (diffusion pseudotime, a commonly used pseudotime method (36)) identified a trajectory of transcriptional states during DLX2-mediated reprogramming across four broad neural cell clusters, in the order of astrocytes, lenti-astrocytes, NPCs, and neuroblasts (FIG. 18A). Pseudotime assumes and reflects the gradual change of the transcriptome, and therefore suggests, but does not prove, the sequential order of cell state progression during DLX2-induced reprogramming. This putative reprogramming trajectory was supported by several lines of evidence. First, cells from the Lenti-DLX2 group expressed the astrocyte-lineage reporter tdT throughout the pseudotime trajectory (FIG. 18A, right top graph), consistent with the lineage-tracing results (FIG. 1D). Second, NPCs and neuroblasts from the Lenti-DLX2 group expressed the lentiviral transgene (FIG. 18A, right mid graph). Interestingly, lentiviral expression decreased as reprogramming progressed, consistent with the immunohistochemistry result (FIG. 3). This is potentially due to epigenetic silencing of the GFAP promoter as cells exit astrocyte fate (FIG. 17B). Third, this putative trajectory was enriched for cells from the Lenti-DLX2 group (FIG. 18A, right bottom graph), consistent with data that neuroblasts were specifically induced by DLX2 but not lentiviral transduction per se (FIG. 1A). Together, scRNA-seq shows that Lenti-DLX2 astrocytes undergo cell state changes towards neuroblasts (Dcx, Calb2) through a proliferating NPC intermediate, which is marked by the expression of Cdk1, Mki67, and Ascl1 (FIG. 17B). Consistent with the lineage-tracing results (FIG. 14A), generation of oligodendrocytes from reprogrammed cells is predicted from activation of genes essential for their development in iNPCs such as Olig1 and Olig2 (FIG. 17B).

### Gene Regulatory Networks Underlying DLX2-Mediated Reprogramming

To identify gene programs dynamically regulated during DLX2-induced reprogramming, unsupervised clustering of genes was conducted to identify 12 expression patterns spanning 2,584 variably expressed genes among the Lenti-DLX2 cells (FIG. 18B, Dataset S1). It may be reasoned that these genes were the most interesting, because high variability of gene expression suggests dynamic regulation during reprogramming. These 12 expression patterns further fell into five broad patterns based on the timing of expression along pseudotime: astrocyte genes (clusters 6, 0), lenti-astrocyte genes (clusters 2, 10), transition genes (cluster 3), NPC genes (clusters 9, 7, 1), and neurogenesis genes (clusters 4, 8, 5, 11) (FIG. 18B). Consistently, known gene markers for astrocytes (Id3, Aldh1I1, and Clu), NPCs (Ascl1), cell cycle (Cdk1, Ccna2, and Mki67), and neurogenesis (Dlx1, Dlx2, Dcx, Dlx6os1, and Calb2) were assigned to their expected expression patterns. Notably, gene cluster 5 (neurogenesis genes) showed significant enrichment of putative targets of Dlx1/2 in the developing mouse brain (37) (p=7.37e-35, Chi-square test, FIG. 17B, bottom left), suggesting that DLX2 drives a similar regulatory program during reprogramming of astrocyte as in neural development.

To understand the functional relevance of these gene clusters, Gene Ontology (GO) enrichment analysis was performed for each cluster (FIG. 18B-GO terms, Dataset S2). First, astrocyte genes (clusters 6, 0) showed enrichment for metabolic terms like lipid metabolic process and glycogen metabolic process, suggesting a metabolic shift during astrocyte conversion. Second, lenti-astrocyte genes (clusters 2, 10) were enriched for immune responses (cluster 10), likely a response to lentiviral infection. In addition, lipid metabolism-related terms were again enriched (cluster 2), whose expression would later be silenced based on the expression pattern of cluster 2, further pointing to a metabolic shift. Third, transition genes (clusters 3) exhibited enrichment for terms related to metabolism in mitochondria, including mitochondrial translation, mitochondrial respiratory chain complex I assembly, and electron transport (Dataset S2). Since neurons, but not astrocytes, use oxidative phosphorylation as their main energy source (38), these data suggest that DLX2-transduced cells use cluster 3 genes to prepare their mitochondria for a switch to oxidative phosphorylation. Fourth, the NPC-enriched genes (clusters 9, 7, 1) were dominated by those involved in cell cycle. Cluster 9 genes were involved in G1/S phase (DNA replication, DNA repair, etc.), while cluster 7 genes represented G2/M phase (cell division, mitotic cytokinesis, etc.). In addition to cell cycle-related terms, cluster 1 was also enriched for RNA processing terms, suggesting that cells used cluster 1 genes to cope with the increased need for RNA synthesis during active cell cycles. Lastly, neurogenesis genes (cluster 4, 8, 5, 1) were enriched in terms related to neural development, which notably included Dcx, Dlx1/2, Dlx6os1, and Calb2 (cluster 5). Together, these results suggested that distinct gene regulatory programs, such as neurogenesis, metabolism, and immune responses, are coordinately regulated during DLX2-mediated reprogramming.

Finally, co-expression and regulatory relationships were used to construct gene regulatory networks (pySCENIC) (39) underlying DLX2-mediated reprogramming. 430 regulons were identified, each representing one transcription factor (TF) and its putative target genes. Distinct patterns of regulon activity was observed (derived from expression levels of component genes) along the reprogramming trajectory (FIG. 18B). High activity score suggested activation, and low activity score suggested repression, of the regulon. For example, known neuronal regulators including Arx, Dix1, Dix5, and Klf7 were specifically active in neuroblasts. Consistent with published data, the Dix1 regulon was significantly enriched in putative Dlx1/2 targets derived from perturbation and ChIP-Seq (37) (FIG. 17B). Helt, a TF required for development of GABAergic neurons (40), was active in non-cycling NPCs. Ascl1 was similarly active in non-cycling NPCs (30). In addition, known regulators of cell cycle (e.g. E2f1) and DNA repair (e.g. Brca1) were specifically active in NPCs. Supporting the notion that reprogrammed astrocytes shift their metabolism from lipid metabolism to oxidative phosphorylation from the gene clustering analysis, two lipid metabolism regulators, Srebf1 and Ppara (41, 42), showed specific activity in astrocytes that decreases during reprogramming. Finally, some regulon-governing TFs potentially work synergistically. For example, small MAF proteins, including MAFG, have been shown to dimerize with CNC protein family members, such as NRF1, NRF2 (whose precursors are NFE2L1 and NFE2L2, respectively) and BACH1 (43). Mafg, Nfe211, and Nfe212 regulons were identified as lenti-astrocyte-specific, suggesting that their interaction may play a role in reprogramming.

### Neurogenic Reprogramming of Resident Astrocytes by DLX2-Induced Regulons

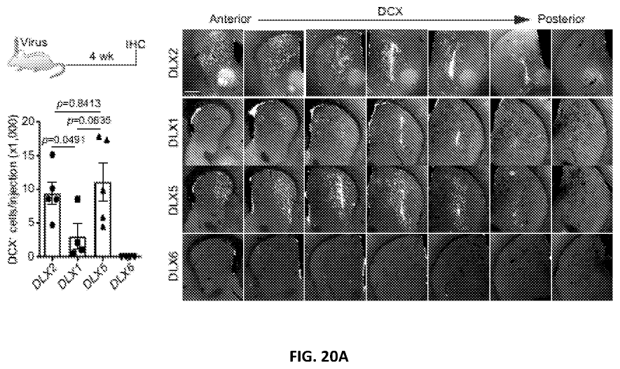

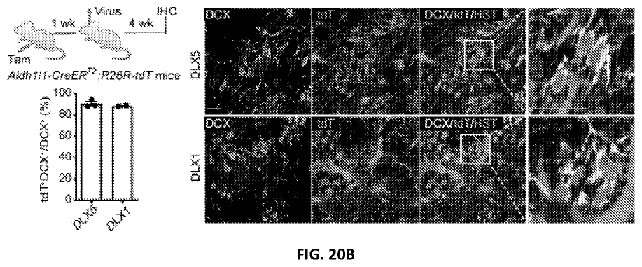

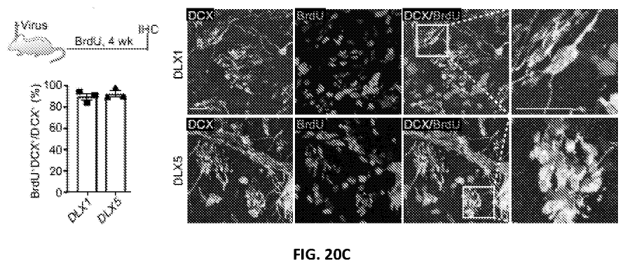

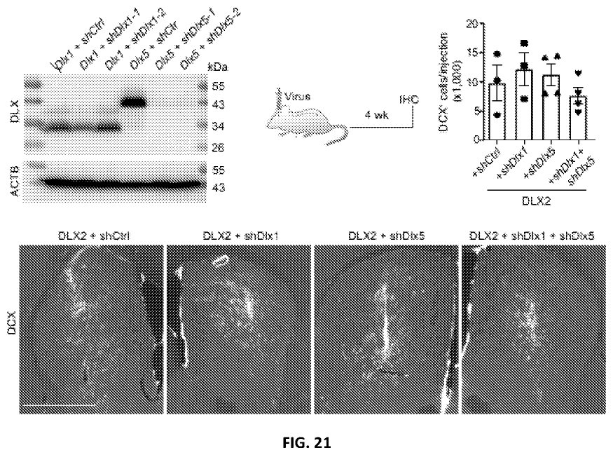

The DLX family was focused on to examine a potential role of these identified regulons, since DLX2 induced not only its endogenous counterpart but also Dlx1 and Dlx5 (FIG. 18B). Interestingly, DLX2, DLX1, and DLX5 shared binding properties in ganglionic eminences (37). As a negative control, DLX6 was also included, which is primarily expressed in differentiated cells (44). Lentiviruses encoding these factors were then individually delivered into the striatum (FIG. 20A). When analyzed at 4 wpv, DLX5 induced more than 10,000 DCX+ cells surrounding the injected region, comparable to that induced by DLX2 (FIG. 20A). DLX1 also induced many DCX+ cells, albeit to a lesser degree. In contrast, no DCX+ cells were observed in DLX6-injected brains (FIG. 20A). To confirm the astrocyte-origin, the Tam-treated Aldh1I1-CreERT2; R26R-tdT mice was employed (FIG. 20B). 88.73% of DCX+ cells induced by DLX1 or DLX5 could be genetically labeled by tdT, indicating an origin of resident astrocytes (FIG. 20B). Cell proliferation assays further showed that 89.71-92.15% of these DLX1- or DLX5-induced DCX+ cells incorporated BrdU (FIG. 20C), suggesting that they passed through a proliferative progenitor state. To determine whether endogenous Dlx1 and/or Dlx5 were also required for the reprogramming activity of ectopic DLX2, their expression through shRNAs was knocked down (FIG. 21). Nonetheless, downregulation of either Dlx1, Dlx5, or their combination failed to significantly change the number of DLX2-induced DCX+ cells (FIG. 21), suggesting that DLX2, DLX1, and DLX5 may play redundant roles during reprogramming.

### DLX2-Mediated Reprogramming Requires Suppression of Notch Signaling

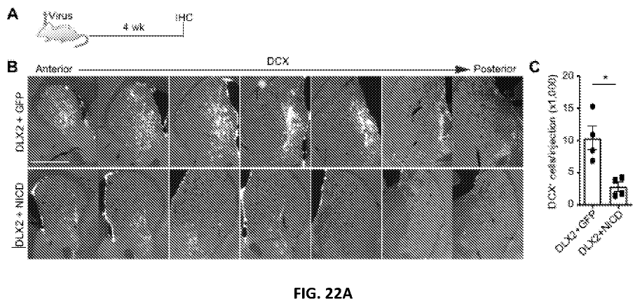

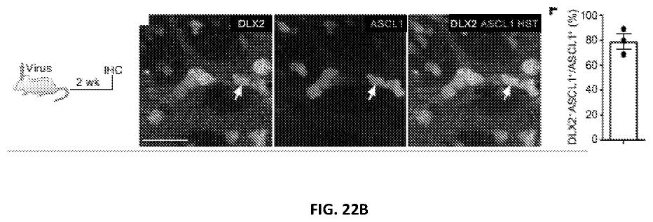

In addition to the DLX family regulons, Notch signaling was also examined since it is highly active in astrocytes and its downregulation is sufficient to initiate a neurogenic program (13-15). Consistently, both Notch1 and Notch2 were detected in cluster 0 astrocytes but not in other cell clusters during DLX2-mediated reprogramming (FIG. 18B; Dataset S1). The cleaved Notch1 intracellular domain (NICD) was used to assess the role of Notch signaling. Adult mice were intracerebrally injected with a mixture of DLX2 virus and a virus expressing GFP (as a control) or NICD under the hGFAP promoter (FIG. 22A). Confirming previous results, DCX+ cells could be abundantly detected in the striatum injected with DLX2 and GFP viruses (FIG. 22A). In contrast, co-expression of NICD with DLX2 significantly reduced the number of DCX+ cells (FIG. 22A, C; p=0.0286 by two-tailed t-test), indicating that suppression of Notch signaling is required for DLX2 to induce reprogramming. To determine whether DLX2-mediated reprogramming is cell autonomous, expression of DLX2 and the induced ASCL1 at 2 wpv was analyzed (FIG. 22B). DLX2 was detectable in approximately 80% of ASCL1+ cells (FIG. 22B), suggesting that DLX2 cell-autonomously induced ASCL1 expression and initiated subsequent cell fate reprogramming. Such a result is consistent with the notion that DLXs promotes proneural programs through repressing Notch signaling (37).

### The DLX2-Mediated Reprogramming Process Resembles Endogenous Neurogenesis

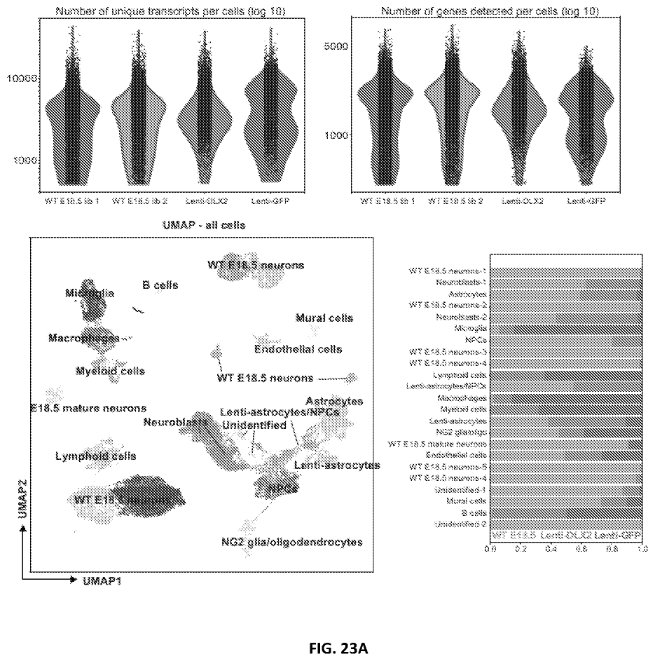

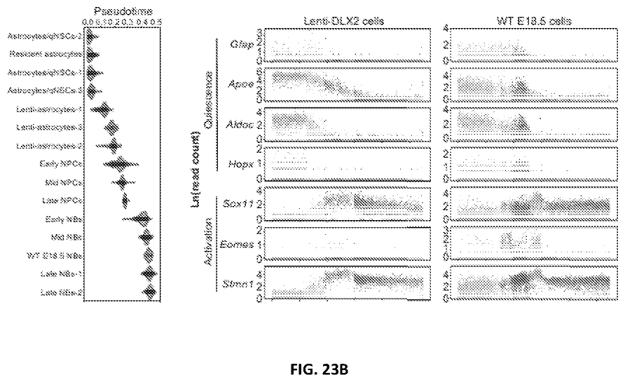

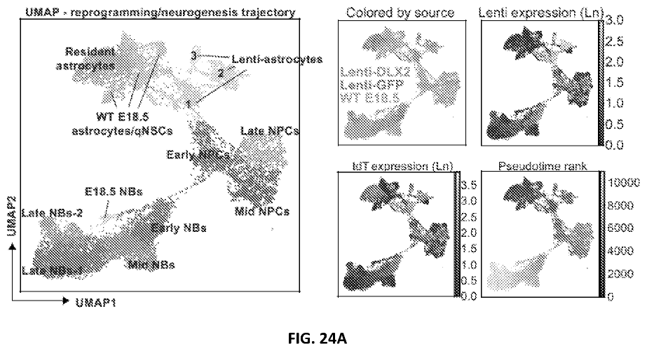

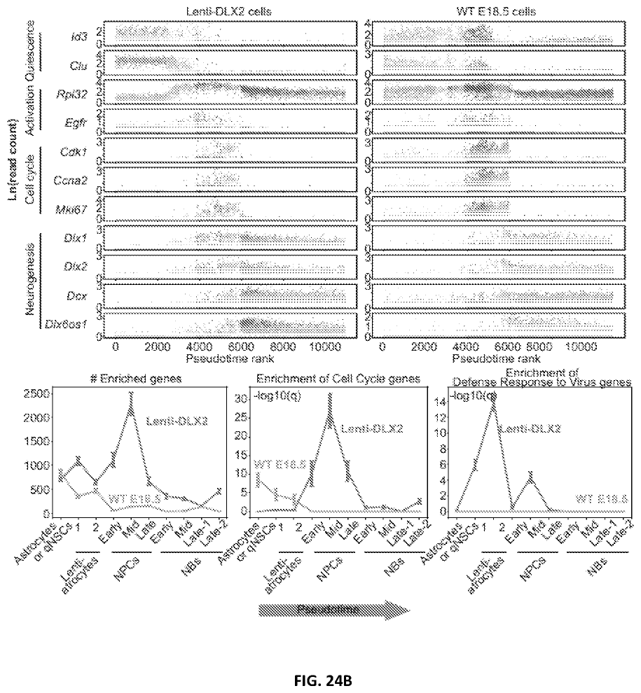

Like endogenous neurogenesis, DLX2-induced reprogramming transitions through an NPC state. In order to study if these two processes also resemble each other transcriptionally publicly available scRNA-seq maps, various factors were integrated from wildtype embryonic day 18.5 (E18.5) mouse brains (cortex, hippocampus, and subventricular zone) (GSE93421) for comparisons between DLX2-induced and endogenous neurogenesis. After data quality filtering, this dataset consisted of 5,756 cells from the Lenti-DLX2 group, 4,611 cells from the Lenti-GFP control, and 19,964 cells from the WT E18.5 brains. Single cells from reprogramming and WT E18.5 datasets have comparable number of transcripts and genes detected per cell (FIG. 23A). After batch correction, clustering, dimensionality reduction, and cell type annotation, astrocytes was focused on, lenti-astrocytes, NPCs, and neuroblasts that together form the main reprogramming/neurogenesis trajectory (FIGS. 24A, 23B).

The gene expression programs were compared in DLX2-induced and endogenous neurogenesis based on pseudotime (FIGS. 24A, 23B). Four groups of genes were previously identified to be sequentially regulated during adult neurogenesis in the mouse SVZ (45). They are involved in NSC quiescence, activation, cell cycle, and neurogenesis. striking parallels were observed in the activation states of these gene sets between DLX2-induced and endogenous embryonic neurogenesis (FIG. 24B). Two cohorts of genes were also examined defining NSC quiescence and activation during adult hippocampal neurogenesis (46) and found that they were similarly dynamically regulated with the progression of DLX2-mediated astrocyte reprogramming (FIG. 23B). Such analyses of stereotypical marker genes clearly show that DLX2-induced astrocytes activate molecular processes typically found in endogenous quiescent NSCs (qNSCs). Interestingly, the repression of quiescence genes (Id3 and Clu) and induction of activation genes (Rpl32) occurs abruptly in reprogramming, in contrast to the gradual changes observed in E18.5 neurogenesis (FIG. 24B). These results suggested that DLX2-induced neurogenesis started from a stable quiescent cell state.

To identify differences between induced and endogenous neurogenesis, differential gene expression analysis was performed between cells from the DLX2 group and WT E18.5 brain cells (Dataset S3). It was observed that the number of differentially expressed genes (DEGs) was highest in the first half of the trajectory (up until mid NPCs), and dropped dramatically as cells entered the neuroblast state (FIG. 24B, left). This indicated that induced and endogenous neurogenesis transcriptionally differ in the early stage but converge towards neurogenesis. This difference in early stage is also reflected by the segregation between astrocytes from reprogramming and astrocytes/qNSCs from E18.5 datasets (FIG. 24A). Two examples of difference were noteworthy. First, cells undergoing endogenous neurogenesis expressed cell cycle genes earlier in pseudotime (states 1-3, NSCs/astrocytes) when compared to cells undergoing DLX2-induced neurogenesis (states 4-6, NPCs) (FIG. 24B, bottom middle). This was in accordance with the observation above that reprogrammed astrocytes went through a molecular transition resembling activation of qNSCs, whereas WT E18.5 brain cells started from an activated NSC state. Second, early in pseudotime, cells undergoing induced neurogenesis activated the expression of viral defense genes (FIG. 24B, bottom right), consistent with the previous observation (FIG. 18B, cluster 10). Interestingly, this immune response declined towards the NPC state. Taken together, differential expression analysis reveals that the difference between induced and endogenous neurogenesis is in part due to cell cycle activation and early responses to viral transduction in reprogrammed cells.

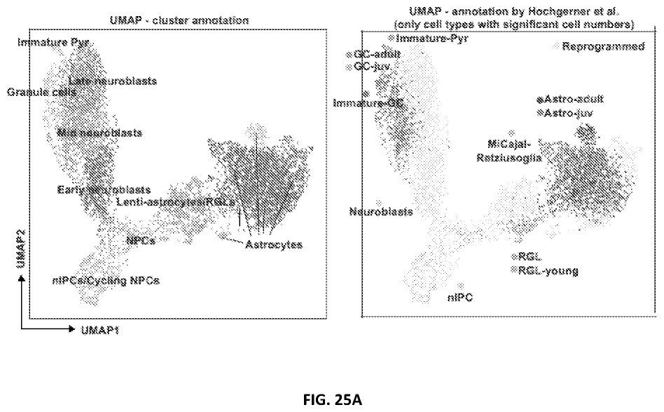

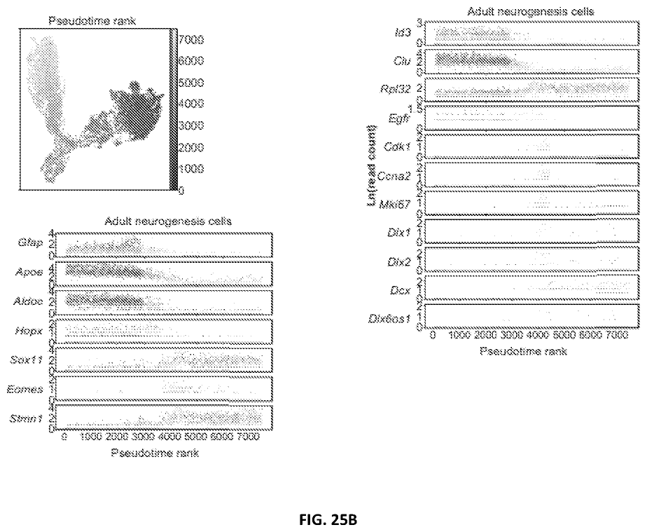

Given the observed differences, one might reason that DLX2-induced reprogramming in adult mice might resemble adult neurogenesis more than embryonic neurogenesis, since adult neurogenesis similarly starts from a quiescent cell state. To test this hypothesis, the reprogramming dataset was integrated with a scRNA-seq dataset of adult neurogenesis in the hippocampus (GSE104323) (47). Only cells from P18, P19, P23, P120, and P132 mice in the adult neurogenesis dataset were used for analysis. After data quality filtering, batch correction, clustering, dimensionality reduction, and selecting only relevant cell types that comprise the main trajectory, this dataset consisted of 4,039 cells from the Lenti-DLX2 group, 182 cells from the Lenti-GFP control (because the reprogramming trajectory is depleted of control group cells), and 3,250 cells from the adult hippocampus (FIG. 25A). Despite differences in platform and processing pipelines, it was observed that adult neurogenesis aligns well with DLX2-induced reprogramming. Notably, astrocytes from the two datasets co-cluster (FIG. 25A), suggesting that the starting cell states are similar. After calculating diffusion pseudotime (FIG. 25B, top left), established neurogenesis genes in adult neurogenesis cells were similarly examined (FIG. 25B). All examined genes followed the expected expression patterns and resembled DLX2-induced reprogramming more than E18.5 neurogenesis. For example, quiescence and activation genes also show an abrupt change (Id3, Clu, and Rpl32 in FIG. 25B right panel, all genes in FIG. 25B). Taken together, these analyses suggest that DLX2-induced reprogramming transcriptionally resembles endogenous neurogenesis in both adult and embryonic stages.

### Example 4. Discussion

The results of this study showed that parenchymal astrocytes can be in vivo reprogrammed by a single transcription factor into INPCs in the adult mouse striatum. Genetic lineage tracings not only confirmed the astrocyte origin but also revealed the multilineage differentiation potential of iNPCs. The in vivo reprogramming process, revealed by scRNA-seq and pseudotime cell trajectories, largely resembled endogenous neurogenesis from NSCs.

scRNA-seq analysis identified four major intermediate cell states during the early reprogramming process: astrocytes, lenti-astrocytes, NPCs, and neuroblasts. First, astrocytes can be roughly divided into “resident astrocytes” and “astrocytes/qNSCs”, with the former exclusively from the Lenti-DLX2 group and latter from WT E18.5 brains. This was consistent with the fact that reprogramming starts from mature parenchymal astrocytes, whereas endogenous neurogenesis was from NSCs. Second, NPCs can be further divided into three clusters: early-, mid-, and late-NPCs. While all express NPC markers such as Ascl1, they differ in gene expression involved in cell cycle and neurogenesis. Such results suggested that the reprogrammed cells sequentially activate NPC genes, cell cycle genes, and neurogenesis genes. Lastly, neuroblasts can also be divided into early-, mid-, and late-neuroblast clusters. Markers for mature neurons such as Calb2 were increasingly expressed from early to mid to late neuroblasts, indicating that cell maturation underlies the distinction between these three neuroblast clusters.

Transient induction of Ascl1 offered a unique opportunity to follow the fates of the reprogrammed astrocytes through genetic lineage tracing in mice with the knockin allele of Ascl1-CreERT2. The time-course lineage tracing revealed that the reprogrammed astrocytes became multipotent iNPCs, giving rise to neuroblasts, neurons, astrocytes, and oligodendrocytes. Such a property of induced ASCL1⁺ cells was consistent with previous reports showing that ASCL1 marks NSCs as well as intermediate progenitors in the adult neurogenic niches (30). Although ASCL1 alone was insufficient to reprogram adult resident striatal astrocytes (48), it may be an essential mediator of DLX2 activity (17). Going forward, it will be of interest to determine the mechanism by which DLX2 induces ASCL1 expression in this reprogramming context. Without being bound by any one theory, a possibility is that DLX2 represses the Notch pathway, which in turn derepresses proneural programs (13-15). Supporting this possibility was the result showing that NICD, the constitutively active form of Notch1, can suppress DLX2-mediated reprogramming. Interestingly, staining data showed that DLX2 and ASCL1 are largely localized in the same cells during the early stage of reprogramming, suggesting that DLX2 directly or indirectly (through Notch suppression) induced ASCL1 expression in a cell-autonomous manner.

DLX2-induced reprogramming strikingly mirrors the regulation of key genes during adult neurogenesis in the SVZ and the hippocampus (45, 46). In contrast, E18.5 neurogenesis has notable differences: quiescence genes (e.g. Clu) are partially silenced, and activation genes (e.g. Rpl32) are partially activated in the beginning of the trajectory. Therefore, DLX2-induced reprogramming seems to resemble adult neurogenesis more than E18.5 neurogenesis. Without being bound to any one theory, one possible explanation observation is that reprogramming in the adult mouse brain was performed.

The reconfiguration of gene regulatory networks revealed metabolic switching and immune responses during reprogramming. Astrocytes used glycolysis, whereas neurons use oxidative phosphorylation, as the main energy source (38). Accordingly, genes involved in the mitochondrial respiratory chain and oxidative phosphorylation are expressed in early NPC states (cluster 3), supporting that metabolism is a potential driving force and can be harnessed for reprogramming (8). Consistently, known metabolic regulators such as Foxk2, Ppara, Epas1, and Srebf1 are activated during reprogramming. The analysis also shows that immune response-related genes (cluster 10) are dynamically regulated during the reprogramming progress. Upon downregulation of astrocytes genes (clusters 0 and 5), cluster 10 genes are upregulated in the reprogramming initiation stage but downregulated quickly as cells enter NPC stage. Although it is well-known that astrocytes can be fast-activated in response to infections and injuries within 2 days (49, 50), such responses are unlikely the cause for the changes on immune response-related genes, since the scRNA-seq analysis was performed 4 weeks post virus injections, a time point that should not acutely reflect the immune response activation. Alternatively, astrocytes as antigen presenting cells in the CNS might need to change their immunological function once converted to neurons (51). Without being bound to any one theory, it is possible that regulation of immune genes may actively contribute to the reprogramming process. As such, DLX2-induced reprogramming was not only neurogenesis/gliogenesis, but also a continuous and precise process involving the temporal regulation of metabolism and immunological events. The potential cross-talks among them require further investigation.

Of note, it was previously claimed that AAV-mediated expression of DLX2, when combined with NEUROD1, could efficiently covert striatal astrocytes into medium spiny neurons (52). However, such a claim was purely based on the virus-expressed reporter and couldn't be confirmed by the more stringent genetic lineage tracing methods (11). Leaky expression of the viral reporter in preexisting neurons accounted for what was claimed to be converted from resident astrocytes (11). Such leaky neuronal expression of the viral reporter was also observed for lentivirus-mediated expression (12). In contrast, the disclosed study has systematically examined each step of the reprogramming process through genetic lineage tracings. Such a thorough analysis unexpectedly revealed multipotentiality of the lentiviral DLX2-reprogrammed astrocytes. An unresolved question is why astrocyte-reprogramming was not observed when employing the AAV system (11). Without being bound to any one theory, one possibility might be AAV-induced cell toxicity (53). It was recently shown that AAV caused rapid and persistent death of NPCs and immature neurons. Another possibility might be the differential cellular responses induced by the DNA virus AAV and the RNA virus lentivirus. These virus-induced cellular responses might contribute to fate reprogramming in vivo.

It is long known that radial glia in the adult neurogenic niches are NSCs, whereas resident parenchymal astrocytes do not have such a property (1, 54, 55). The study identified a genetic switch that can turn on the latent multipotentiality of mature parenchymal astrocytes, underscoring their extreme plasticity and a potential for providing all neural cell types that are needed for regenerative medicine.

### Example 5. Summary and Scientific Significance

Astrocytes in the adult brain show cellular plasticity; however, whether they have the potential to generate multiple lineages remains unclear. Here, in vivo screens were performed and DLX2 was identified as a transcription factor that can unleash the multipotentiality of adult resident astrocytes. Genetic lineage tracing and time-course analyses revealed that DLX2 enables astrocytes to rapidly become ASCL1⁺ neural progenitor cells, which give rise to neurons, astrocytes, and oligodendrocytes in the adult mouse striatum. Single-cell transcriptomics and pseudotime trajectories further confirmed a neural stem cell-like behavior of reprogrammed astrocytes, transitioning from quiescence to activation, proliferation, and neurogenesis. Gene regulatory networks and mouse genetics identified and confirmed key nodes mediating DLX2-dependent fate reprogramming. These included activation of endogenous DLX family transcription factors and suppression of Notch signaling. Such reprogramming-induced multipotency of resident glial cells may be exploited for neural regeneration.

Outside the neurogenic niches, the adult brain lacks multipotent progenitor cells. In this study, a series of in vivo screens were performed and revealed that a single factor can induce resident brain astrocytes to become neural progenitor cells (iNPCs), which then generated neurons, astrocytes and oligodendrocytes. Such a conclusion was supported by scRNA-seq and multiple lineage-tracing experiments. The instant discovery of iNPCs was fundamentally important for regenerative medicine since neural injuries or degeneration often leads to loss/dysfunction of all three neural lineages. The instant findings also provided novel insights into cell plasticity in the adult mammalian brain, which has largely lost the regenerative capacity.

### REFERENCES CITED

References associated with experimental procedures:
1. Niu W, et al. (2015) SOX2 reprograms resident astrocytes into neural progenitors in the adult brain. Stem Cell Reports 4 (5): 780-794.
2. Niu W, et al. (2013) In vivo reprogramming of astrocytes to neuroblasts in the adult brain. Nat Cell Biol 15 (10): 1164-1175.
3. Su Z, Niu W, Liu M L, Zou Y, & Zhang C L (2014) In vivo conversion of astrocytes to neurons in the injured adult spinal cord. Nat Commun 5:3338.
4. Wang L L, et al. (2016) The p53 Pathway Controls SOX2-Mediated Reprogramming in the Adult Mouse Spinal Cord. Cell Rep 17 (3): 891-903.
5. Wolf F A, Angerer P, & Theis F J (2018) SCANPY: large-scale single-cell gene expression data analysis. Genome Biol 19 (1): 15.
6. Korsunsky I, et al. (2019) Fast, sensitive and accurate integration of single-cell data with Harmony. Nat Methods 16 (12): 1289-1296.
7. Van de Sande B, et al. (2020) A scalable SCENIC workflow for single-cell gene regulatory network analysis. Nat Protoc 15 (7): 2247-2276.

8 Lindtner S, et al. (2019) Genomic Resolution of DLX-Orchestrated Transcriptional Circuits Driving Development of Forebrain GABAergic Neurons. Cell Rep 28 (8): 2048-2063 e2048.
9. Klopfenstein D V, et al. (2018) GOATOOLS: A Python library for Gene Ontology analyses. Sci Rep 8 (1): 10872.

### REFERENCE CITED IN RESULTS AND DISCUSSION

1. Kriegstein A & Alvarez-Buylla A (2009) The glial nature of embryonic and adult neural stem cells. Annu Rev Neurosci 32:149-184.
2. Goncalves J T, Schafer S T, & Gage F H (2016) Adult Neurogenesis in the Hippocampus: From Stem Cells to Behavior. Cell 167 (4): 897-914.
3. Gotz M, Nakafuku M, & Petrik D (2016) Neurogenesis in the Developing and Adult Brain-Similarities and Key Differences. Cold Spring Harb Perspect Biol 8 (7).
4. Lledo P M, Alonso M, & Grubb M S (2006) Adult neurogenesis and functional plasticity in neuronal circuits. Nat Rev Neurosci 7 (3): 179-193.
5. Zhang C L, Zou Y, He W, Gage F H, & Evans R M (2008) A role for adult TLX-positive neural stem cells in learning and behaviour. Nature 451 (7181): 1004-1007.
6. Barker R A, Gotz M, & Parmar M (2018) New approaches for brain repair-from rescue to reprogramming. Nature 557 (7705): 329-334.
7. Tai W, Xu X M, & Zhang C L (2020) Regeneration Through in vivo Cell Fate
   Reprogramming for Neural Repair. Front Cell Neurosci 14:107.
8. Gascon S, Masserdotti G, Russo G L, & Gotz M (2017) Direct Neuronal Reprogramming: Achievements, Hurdles, and New Roads to Success. Cell Stem Cell 21 (1): 18-34.
9. Wang L-L & Zhang C-L (2018) Engineering new neurons: in vivo reprogramming in mammalian brain and spinal cord. Cell and Tissue Research 371 (1): 201-212.
10. Wang L-L, Garcia C S, Zhong X, Ma S, & Zhang C-L (2020) Rapid and efficient <em> in vivo</em>astrocyte-to-neuron conversion with regional identity and connectivity? bioRxiv: 2020.2008.2016.253195.
11. Wang L-L, et al. (2021) Revisiting astrocyte to neuron conversion with lineage tracing in vivo. Cell 184 (21): 5465-5481.e5416.
12. Rao Y, et al. (2021) NeuroD1 induces microglial apoptosis and cannot induce microglia-to-neuron cross-lineage reprogramming. Neuron 109 (24): 4094-4108.e4095.
13. Magnusson J P, et al. (2014) A latent neurogenic program in astrocytes regulated by Notch signaling in the mouse. Science 346 (6206): 237-241.
14. Magnusson J P, et al. (2020) Activation of a neural stem cell transcriptional program in parenchymal astrocytes. Elife 9.
15. Zamboni M, Llorens-Bobadilla E, Magnusson J P, & Frisen J (2020) A Widespread Neurogenic Potential of Neocortical Astrocytes Is Induced by Injury. Cell Stem Cell 27 (4): 605-617 e605.
16. Nato G, et al. (2015) Striatal astrocytes produce neuroblasts in an excitotoxic model of Huntington's disease. Development 142 (5): 840-845.
17. Niu W, et al. (2015) SOX2 reprograms resident astrocytes into neural progenitors in the adult brain. Stem Cell Reports 4 (5): 780-794.
18. Niu W, et al. (2013) In vivo reprogramming of astrocytes to neuroblasts in the adult brain. Nat Cell Biol 15 (10): 1164-1175.
19. Torper O, et al. (2013) Generation of induced neurons via direct conversion in vivo. Proc Natl Acad Sci USA 110 (17): 7038-7043.
20 Grande A, et al. (2013) Environmental impact on direct neuronal reprogramming in vivo in the adult brain. Nat Commun 4:2373.
21. Heinrich C, et al. (2014) Sox2-mediated conversion of NG2 glia into induced neurons in the injured adult cerebral cortex. Stem Cell Reports 3 (6): 1000-1014.
22. Tai W, et al. (2021) In vivo reprogramming of NG2 glia enables adult neurogenesis and functional recovery following spinal cord injury. Cell Stem Cell.
23. Su Z, Niu W, Liu M L, Zou Y, & Zhang C L (2014) In vivo conversion of astrocytes to neurons in the injured adult spinal cord. Nat Commun 5:3338.
24. Jorstad N L, et al. (2017) Stimulation of functional neuronal regeneration from Müller glia in adult mice. Nature 548 (7665): 103-107.
25. Hoang T, et al. (2020) Gene regulatory networks controlling vertebrate retinal regeneration. Science 370 (6519).
26. Gascón S, et al. (2016) Identification and Successful Negotiation of a Metabolic Checkpoint in Direct Neuronal Reprogramming. Cell Stem Cell 18 (3): 396-409.
27. Lentini C, et al. (2021) Reprogramming reactive glia into interneurons reduces chronic seizure activity in a mouse model of mesial temporal lobe epilepsy. Cell Stem Cell 28 (12): 2104-2121.e2110.
28 Srinivasan R, et al. (2016) New Transgenic Mouse Lines for Selectively Targeting Astrocytes and Studying Calcium Signals in Astrocyte Processes In Situ and In Vivo. Neuron 92 (6): 1181-1195.
29. Kaneko N, et al. (2018) New neurons use Slit-Robo signaling to migrate through the glial meshwork and approach a lesion for functional regeneration. Sci Adv 4 (12): eaav0618.
30. Kim E J, Ables J L, Dickel L K, Eisch A J, & Johnson J E (2011) Ascl1 (Mash1) defines cells with long-term neurogenic potential in subgranular and subventricular zones in adult mouse brain. PLOS One 6 (3): e18472.
31. Wang L L, et al. (2016) The p53 Pathway Controls SOX2-Mediated Reprogramming in the Adult Mouse Spinal Cord. Cell Rep 17 (3): 891-903.
32. Arlotta P, Molyneaux B J, Jabaudon D, Yoshida Y, & Macklis J D (2008) Ctip2 controls the differentiation of medium spiny neurons and the establishment of the cellular architecture of the striatum. J Neurosci 28 (3): 622-632.
33. Revishchin A V, Okhotin V E, Korochkin L I, & Pavlova G V (2010) A new population of calretinin-positive cells, presumptively neurons, with polymorphous spines in the mouse forebrain. Neurosci Behav Physiol 40 (5): 541-552.
34. Korsunsky I, et al. (2019) Fast, sensitive and accurate integration of single-cell data with Harmony. Nat Methods 16 (12): 1289-1296.
35. Wolf F A, et al. (2019) PAGA: graph abstraction reconciles clustering with trajectory inference through a topology preserving map of single cells. Genome Biol 20 (1): 59. Haghverdi L, Buttner M, Wolf F A, Buettner F, & Theis F J (2016) Diffusion pseudotime 36. robustly reconstructs lineage branching. Nat Methods 13 (10): 845-848.
37. Lindtner S, et al. (2019) Genomic Resolution of DLX-Orchestrated Transcriptional Circuits Driving Development of Forebrain GABAergic Neurons. Cell Rep 28 (8): 2048-2063 e2048.
38. Maffezzini C, Calvo-Garrido J, Wredenberg A, & Freyer C (2020) Metabolic regulation of neurodifferentiation in the adult brain. Cell Mol Life Sci 77 (13): 2483-2496.
39 Van de Sande B, et al. (2020) A scalable SCENIC workflow for single-cell gene regulatory network analysis. Nat Protoc 15 (7): 2247-2276.
40. Nakatani T, Minaki Y, Kumai M, & Ono Y (2007) Helt determines GABAergic over glutamatergic neuronal fate by repressing Ngn genes in the developing mesencephalon. Development 134 (15): 2783-2793.
41. Yoon M (2009) The role of PPARalpha in lipid metabolism and obesity: focusing on the effects of estrogen on PPARalpha actions. Pharmacol Res 60 (3): 151-159.
42. Eberle D, Hegarty B, Bossard P, Ferre P, & Foufelle F (2004) SREBP transcription factors: master regulators of lipid homeostasis. Biochimie 86 (11): 839-848.
43. Shavit J A, et al. (1998) Impaired megakaryopoiesis and behavioral defects in mafG-null mutant mice. Genes Dev 12 (14): 2164-2174.
44. Liu J K, Ghattas I, Liu S, Chen S, & Rubenstein J L (1997) Dlx genes encode DNA-binding proteins that are expressed in an overlapping and sequential pattern during basal ganglia differentiation. Dev Dyn 210 (4): 498-512.
45. Dulken B W, Leeman D S, Boutet S C, Hebestreit K, & Brunet A (2017) Single-Cell Transcriptomic Analysis Defines Heterogeneity and Transcriptional Dynamics in the Adult Neural Stem Cell Lineage. Cell Rep 18 (3): 777-790.
46. Shin J, et al. (2015) Single-Cell RNA-Seq with Waterfall Reveals Molecular Cascades underlying Adult Neurogenesis. Cell Stem Cell 17 (3): 360-372.
47. Hochgerner H, Zeisel A, Lonnerberg P, & Linnarsson S (2018) Conserved properties of dentate gyrus neurogenesis across postnatal development revealed by single-cell RNA sequencing. Nat Neurosci 21 (2): 290-299.
48. Islam M M, et al. (2015) Enhancer Analysis Unveils Genetic Interactions between TLX and SOX2 in Neural Stem Cells and In Vivo Reprogramming. Stem Cell Reports 5 (5): 805-815.
49. Kim J V & Dustin M L (2006) Innate response to focal necrotic injury inside the blood-brain barrier. J Immunol 177 (8): 5269-5277.
50. Rinaman L, Card J P, & Enquist L W (1993) Spatiotemporal responses of astrocytes, ramified microglia, and brain macrophages to central neuronal infection with pseudorabies virus. J Neurosci 13 (2): 685-702.
51. Dong Y & Benveniste E N (2001) Immune function of astrocytes. Glia 36 (2): 180-190.
52. Wu Z, et al. (2020) Gene therapy conversion of striatal astrocytes into GABAergic neurons in mouse models of Huntington's disease. Nature Communications 11 (1): 1105.
53. Johnston S, et al. (2021) AAV ablates neurogenesis in the adult murine hippocampus. Elife 10.
54. Doetsch F, Caille I, Lim D A, Garcia-Verdugo J M, & Alvarez-Buylla A (1999) Subventricular zone astrocytes are neural stem cells in the adult mammalian brain. Cell 97 (6): 703-716.
55. Robel S, Berninger B, & Gotz M (2011) The stem cell potential of glia: lessons from reactive gliosis. Nat Rev Neurosci 12 (2): 88-104.

What is claimed is:

1. A viral vector comprising a polynucleotide sequence comprising a nucleic acid sequence encoding one or more of a DLX family transcription factor or derivatives, variants or fragment thereof operably linked to a glial cell targeting promoter.

2. The viral vector of claim 1, wherein the viral vector is not an adeno-associated viral vector (AAV).

3. The viral vector of claim 1, wherein the viral vector is selected from a group consisting of a retrograde virus, retrovirus, herpesvirus, lentivirus, poxvirus, or papiloma virus vector.

4. The viral vector of claim 1, wherein the viral vector is an RNA viral vector.

5. The viral vector of claim 4, wherein the viral vector is a lentiviral vector.

6. The viral vector of claim 1, wherein the glial cell targeting promoter is selected from any one of hGFAP, hgfa2, hALDH1L1, hgfa28, hgfaABC₁D hNG2, hIBA1, hCD68, hPDGFRA, or hPDGFRB.

7. The viral vector of claim 1, wherein the polynucleotide sequence further comprises one or more regulatory sequences selected from an enhancer, a leader, a transcription start site (TSS), a linker, 5′ and 3′ untranslated regions (UTRs), an intron, a polyadenylation signal, and a termination region or sequence.

8. A viral vector comprising a polynucleotide sequence comprising a nucleic acid sequence at least 60% identical to any one of SEQ ID. NOS. 1-6.

9. A composition comprising the viral vector of any one of claims 1-8 and a pharmaceutically acceptable excipient.

10. A composition comprising an effective amount of a DLX family transcription factor or derivatives, variants, or fragment thereof, and a pharmaceutically acceptable carrier.

11. The composition of claim 10, wherein the DLX family transcription factor is DLX2.

12. A composition comprising an effective amount of an RNA encoding a polypeptide corresponding to a DLX family transcription factor or derivatives, variants, or fragment thereof and a pharmaceutically acceptable excipient.

13. The composition of claim 12, wherein the polypeptide comprises an amino acid sequence of any one of SEQ ID NOS. 7-12.

14. A method for inducing neural regeneration in a subject in need thereof, the method comprising administering to the subject the composition of any one of claims 9-13.

15. The method of claim 14, wherein the subject is a mammal.

16. The method of claim 15, wherein the subject is a human.

17. The method of claim 14, wherein the wherein the administering is by any one of an intravenous, intracranial, intrathecal, subcutaneous, or intranasal route.

18. A method for inducing multilineage reprogramming of glial cells, the method comprising contacting the cell with a composition comprising the viral vector of any one of claims 1-7.

19. A method for inducing multilineage reprogramming of glial cells, the method comprising contacting the cell with a composition comprising any one of claims 9-13.

20. A method for inducing neural regeneration in a subject in need thereof, the method comprising administering to the subject a composition comprising a therapeutically effective amount of a polynucleotide sequence comprising a nucleic acid sequence at least 60% identical to any one of SEQ ID. NOS. 1-6, 13-17.

21. The method of claim 20, wherein the polynucleotide sequence is packaged within a viral vector, wherein the viral vector is not an AAV.

22. The method of claim 20, wherein the viral vector is selected from a group consisting of adenovirus, a retrograde virus, retrovirus, herpesvirus, lentivirus, poxvirus, or papiloma virus.

23. The method of claim 20, wherein the composition further comprises a pharmaceutically acceptable excipient.

24. The method of claim 20, wherein the subject is a mammal.

25. The method of claim 24, wherein the subject is a human.

26. The method of claim 20, wherein the wherein the administering is by any one of an intravenous, intracranial, intrathecal, subcutaneous, or intranasal route.

27. The method of claim 20, wherein the subject is suspected of, predicted to or diagnosed with a neural injury, or a neurodegenerative disease.

28. The method of claim 27, wherein the neurodegenerative disease Alzheimer's disease, Parkinson's disease, amyotrophic lateral sclerosis, multiple sclerosis, epilepsy, seizures or multiple system atrophy.

29. Use of the compositions provided in any one of claims 1-13 for inducing multilineage neural regeneration in a subject in need thereof.

30. Use of the compositions provided in any one of claims 1-13 for treatment of neural injury, or a neurodegenerative disease.


SEQUENCE LISTING (17 sequences)

SEQ ID NO: 1 - DNA, 768 bp, synthetic construct
  source 1..768: mol_type = other DNA
atgaccatgaccaccatgccagaaagtctcaacagccccgtgtcgggcaaggcggtgtttatggagtttgggccgcccaaccagcaaatgtctccttctcccatgtcccacgggcactactccatgcactgtttacactcggcgggccattcgcagcccgacggcgcctacagctcagcctcgtccttctcccgaccgctgggctacccctacgtcaactcggtcagcagccacgcatccagcccctacatcagttcggtgcagtcctacccgggcagcgccagcctcgcccagagccgcctggaggacccaggggcggactcggagaagagcacggtggtggaaggcggtgaagtgcgcttcaatggcaagggaaaaaagatccgtaaacccaggacgatttattgcagtttgcagttgcaggctttgaaccggaggttccagcaaactcagtacctagctctgccggagagggcggagctcgcggcctctttgggactcacacagactcaggtcaagatctggttccaaaacaagcgatccaagttcaagaagctgatgaagcagggtggggcggctctggagggtagtgcgttggccaacggtcgggccctgtctgctggctccccacccgtgccgcccggctggaaccctaactcttcatccgggaagggctcaggaggaaacgcgggctcctatatccccagctacacatcgtggtacccttcagcgcaccaagaagctatgcagcaaccccaacttatgtga

SEQ ID NO: 2 - DNA, 987 bp, synthetic construct
  source 1..987: mol_type = other DNA
atgactggagtctttgacagtctagtggctgatatgcactcgacccagatcgccgcctccagcacgtaccaccagcaccagcagcccccgagcggcggcggcgccggcccgggtggcaacagcagcagcagcagcagcctccacaagccccaggagtcgcccacccttccggtgtccaccgccaccgacagcagctactacaccaaccagcagcacccggcgggcggcggcggcggcgggggctcgccctacgcgcacatgggttcctaccagtaccaagccagcggcctcaacaacgtcccttactccgccaagagcagctatgacctgggctacaccgccgcctacacctcctacgctccctatggaaccagttcgtccccagccaacaacgagcctgagaaggaggaccttgagcctgaaattcggatagtgaacgggaagccaaagaaagtccggaaaccccgcaccatctactccagtttccagctggcggctcttcagcggcgtttccaaaagactcaatacttggccttgccggagcgagccgagctggcggcctctctgggcctcacccagactcaggtcaaaatctggttccagaaccgccggtccaagttcaagaagatgtggaaaagtggtgagatcccctcggagcagcaccctggggccagcgcttctccaccttgtgcttcgccgccagtctcagcgccggcctcctgggactttggtgtgccgcagcggatggcgggcggcggtggtccgggcagtggcggcagcggcgccggcagctcgggctccagcccgagcagcgcggcctcggcttttctgggcaactacccctggtaccaccagacctcgggatccgcctcacacctgcaggccacggcgccgctgctgcaccccactcagaccccgcagccgcatcaccaccaccaccatcacggcggcgggggcgccccggtgagcgcggggacgattttctaa

SEQ ID NO: 3 - DNA, 864 bp, synthetic construct
  source 1..864: mol_type = other DNA
atgagtggctccttcgatcgcaagctcagcagcatcctcaccgacatctccagctcccttagctgccatgcgggctccaaggactcgcctaccctgcccgagtcttctgtcactgacctgggctactacagcgctccccagcacgattactactcgggccagccctatggccagacggtgaacccctacacctaccaccaccaattcaatctcaatgggcttgcaggcacgggcgcttactcgcccaagtcggaatatacctacggagcctcctaccggcaatacggggcgtatcgggagcagccgctgccagcccaggacccagtgtcggtgaaggaggagccggaagcagaggtgcgcatggtgaatgggaagcccaagaaggtccgaaagccgcgtacaatctactccagctaccagctggccgccctgcagcgccgcttccagaaggcccagtacctggcgctgcccgagcgcgccgagctggccgcgcagctgggcctcacgcagacacaggtgaaaatctggttccagaaccgccgttccaagttcaagaaactctacaagaacggggaggtgccgctggagcacagtcccaataacagtgattccatggcctgcaactcaccaccatcacccgccctctgggacacctcttcccactccactccggcccctgcccgcagtcagctgcccccgccgctcccatacagtgcctcccccagctacctggacgaccccaccaactcctggtatcacgcacagaacctgagtggaccccacttacagcagcagccgcctcagccagccaccctgcaccatgcctctcccgggcccccgcccaaccctggggctgtgtactga

SEQ ID NO: 4 - DNA, 723 bp, synthetic construct
  source 1..723: mol_type = other DNA
atgacctctttgccctgccccctccccggccgggacgcctccaaagctgtcttcccagacctcgcccctgtcccgtcggtagcggctgcctacccgcttggcttgtcccctacaaccgcagcctcccccaatttgtcctactccaggccgtatggccacctcctgtcttacccctacaccgagccagcgaaccccggagactcctacctgtcctgccagcaacccgcggcgctctctcagcccctctgcggacctgcagagcaccctcaggaactcgaggcagactcggagaagccgcggctgtccccggaaccctccgagcggcgccctcaggcccccgccaaaaagctccgcaagccgaggaccatctactccagcctgcagctgcagcacctaaaccagcgtttccagcacacgcagtacctggcgctgcccgagagggcccagctggcagcgcagctcggcctcacccagacccaggtaaagatctggtttcagaacaaacgctccaagtataagaagctcctgaagcagaattctggggggcaggaaggggacttccctgggaggaccttctctgtgtctccctgctccccacccctcccctccctctgggatctacccaaggcagggaccctgcccaccagtggctatggcaacagctttggagcctggtatcagcatcactcctcagatgtcctggcttcgcctcagatgatgtga

SEQ ID NO: 5 - DNA, 870 bp, synthetic construct
  source 1..870: mol_type = other DNA
atgacaggagtgtttgacagaagggtccccagcatccgatccggcgacttccaagctccgttccagacgtccgcagctatgcaccatccgtctcaggaatcgccaactttgcccgagtcttcagctaccgattctgactactacagccctacggggggagccccgcacggctactgctctcctacctcggcttcctatggcaaagctctcaacccctaccagtatcagtatcacggcgtgaacggctccgccgggagctacccagccaaagcttatgccgactatagctacgctagctcctaccaccagtacggcggcgcctacaaccgcgtcccaagcgccaccaaccagccagagaaagaagtgaccgagcccgaggtgagaatggtgaatggcaaaccaaagaaagttcgtaaacccaggactatttattccagctttcagctggccgcattacagagaaggtttcagaagactcagtacctcgccttgccggaacgcgccgagctggccgcctcgctgggattgacacaaacacaggtgaaaatctggtttcagaacaaaagatccaagatcaagaagatcatgaaaaacggggagatgcccccggagcacagtcccagctccagcgacccaatggcgtgtaactcgccgcagtctccagcggtgtgggagccccagggctcgtcccgctcgctcagccaccaccctcatgcccaccctccgacctccaaccagtccccagcgtccagctacctggagaactctgcatcctggtacacaagtgcagccagctcaatcaattcccacctgccgccgccgggctccttacagcacccgctggcgctggcctccgggacactctattag

SEQ ID NO: 6 - DNA, 882 bp, synthetic construct
  source 1..882: mol_type = other DNA
atgatgaccatgactacgatggctgacggcttggaaggccaggactcgtccaaatccgccttcatggagttcgggcagcagcagcagcagcagcagcaacagcagcagcagcagcagcagcaacagcaacagccgccgccgccgccgccgccgccgccgcagccgcactcgcagcagagctccccggccatggcaggcgcgcactaccctctgcactgcctgcactcggcggcggcggcggcagcggccggctcgcaccaccaccaccaccaccagcaccaccaccacggctcgccctacgcgtcgggcggagggaactcctacaaccaccgctcgctcgccgcctacccctacatgagccactcgcagcacagcccttacctccagtcctaccacaacagcagcgcagccgcccagacgcgaggggacgacacagatcaacaaaaaactacagtgattgaaaacggggaaatcaggttcaatggaaaagggaaaaagattcggaagcctcggaccatttattccagcctgcagctccaggctttaaaccatcgctttcagcagacacagtatctggcccttccagagagagccgaactggcagcttccttaggactgacacaaacacaggtgaagatatggtttcagaacaaacgctctaagtttaagaaactgctgaagcagggcagtaatcctcatgagagcgaccccctccagggctcggcggccctgtcgccacgctcgccagcgctgcctccagtctgggacgtttctgcctcggccaagggtgtcagtatgccccccaacagctacatgcctggctattctcactggtactcctctccacaccaggacacgatgcagagaccacagatgatgtga

SEQ ID NO: 7 - protein, 255 aa, Homo sapiens
MTMTTMPESLNSPVSGKAVFMEFGPPNQQMSPSPMSHGHYSMHCLHSAGHSQPDGAYSSASSFSRPLGYPYVNSVSSHASSPYISSVQSYPGSASLAQSRLEDPGADSEKSTVVEGGEVRFNGKGKKIRKPRTIYSSLQLQALNRRFQQTQYLALPERAELAASLGLTQTQVKIWFQNKRSKFKKLMKQGGAALEGSALANGRALSAGSPPVPPGWNPNSSSGKGSGGNAGSYIPSYTSWYPSAHQEAMQQPQLM

SEQ ID NO: 8 - protein, 328 aa, Homo sapiens
MTGVFDSLVADMHSTQIAASSTYHQHQQPPSGGGAGPGGNSSSSSSLHKPQESPTLPVSTATDSSYYTNQQHPAGGGGGGGSPYAHMGSYQYQASGLNNVPYSAKSSYDLGYTAAYTSYAPYGTSSSPANNEPEKEDLEPEIRIVNGKPKKVRKPRTIYSSFQLAALQRRFQKTQYLALPERAELAASLGLTQTQVKIWFQNRRSKFKKMWKSGEIPSEQHPGASASPPCASPPVSAPASWDFGVPQRMAGGGGPGSGGSGAGSSGSSPSSAASAFLGNYPWYHQTSGSASHLQATAPLLHPTQTPQPHHHHHHHGGGGAPVSAGTIF

SEQ ID NO: 9 - protein, 287 aa, Homo sapiens
MSGSFDRKLSSILTDISSSLSCHAGSKDSPTLPESSVTDLGYYSAPQHDYYSGQPYGQTVNPYTYHHQFNLNGLAGTGAYSPKSEYTYGASYRQYGAYREQPLPAQDPVSVKEEPEAEVRMVNGKPKKVRKPRTIYSSYQLAALQRRFQKAQYLALPERAELAAQLGLTQTQVKIWFQNRRSKFKKLYKNGEVPLEHSPNNSDSMACNSPPSPALWDTSSHSTPAPARSQLPPPLPYSASPSYLDDPTNSWYHAQNLSGPHLQQQPPQPATLHHASPGPPPNPGAVY

SEQ ID NO: 10 - protein, 240 aa, Homo sapiens
MTSLPCPLPGRDASKAVFPDLAPVPSVAAAYPLGLSPTTAASPNLSYSRPYGHLLSYPYTEPANPGDSYLSCQQPAALSQPLCGPAEHPQELEADSEKPRLSPEPSERRPQAPAKKLRKPRTIYSSLQLQHLNQRFQHTQYLALPERAQLAAQLGLTQTQVKIWFQNKRSKYKKLLKQNSGGQEGDFPGRTFSVSPCSPPLPSLWDLPKAGTLPTSGYGNSFGAWYQHHSSDVLASPQMM

SEQ ID NO: 11 - protein, 289 aa, Homo sapiens
MTGVFDRRVPSIRSGDFQAPFQTSAAMHHPSQESPTLPESSATDSDYYSPTGGAPHGYCSPTSASYGKALNPYQYQYHGVNGSAGSYPAKAYADYSYASSYHQYGGAYNRVPSATNQPEKEVTEPEVRMVNGKPKKVRKPRTIYSSFQLAALQRRFQKTQYLALPERAELAASLGLTQTQVKIWFQNKRSKIKKIMKNGEMPPEHSPSSSDPMACNSPQSPAVWEPQGSSRSLSHHPHAHPPTSNQSPASSYLENSASWYTSAASSINSHLPPPGSLQHPLALASGTLY

SEQ ID NO: 12 - protein, 175 aa, Homo sapiens
MSHSQHSPYLQSYHNSSAAAQTRGDDTDQQKTTVIENGEIRFNGKGKKIRKPRTIYSSLQLQALNHRFQQTQYLALPERAELAASLGLTQTQVKIWFQNKRSKFKKLLKQGSNPHESDPLQGSAALSPRSPALPPVWDVSASAKGVSMPPNSYMPGYSHWYSSPHQDTMQRPQMM

SEQ ID NO: 13 - DNA, 9360 bp, synthetic construct
  source 1..9360: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaattcactttgaattcaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttgagaggagacgcatcacctccgctgctcgcaccggtgccaccatgaccatgaccaccatgccagaaagtctcaacagccccgtgtcgggcaaggcggtgtttatggagtttgggccgcccaaccagcaaatgtctccttctcccatgtcccacgggcactactccatgcactgtttacactcggcgggccattcgcagcccgacggcgcctacagctcagcctcgtccttctcccgaccgctgggctacccctacgtcaactcggtcagcagccacgcatccagcccctacatcagttcggtgcagtcctacccgggcagcgccagcctcgcccagagccgcctggaggacccaggggcggactcggagaagagcacggtggtggaaggcggtgaagtgcgcttcaatggcaagggaaaaaagatccgtaaacccaggacgatttattgcagtttgcagttgcaggctttgaaccggaggttccagcaaactcagtacctagctctgccggagagggcggagctcgcggcctctttgggactcacacagactcaggtcaagatctggttccaaaacaagcgatccaagttcaagaagctgatgaagcagggtggggcggctctggagggtagtgcgttggccaacggtcgggccctgtctgctggctccccacccgtgccgcccggctggaaccctaactcttcatccgggaagggctcaggaggaaacgcgggctcctatatccccagctacacatcgtggtacccttcagcgcaccaagaagctatgcagcaaccccaacttatgtgactcgagggatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgagacctggaaaaacatggagcaatcacaagtagcaacacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 9615 bp, synthetic construct
  source 1..9615: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaattcactttgaattcaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcaccggtgccaccatgactggagtctttgacagtctagtggctgatatgcactcgacccagatcgccgcctccagcacgtaccaccagcaccagcagcccccgagcggcggcggcgccggcccgggtggcaacagcagcagcagcagcagcctccacaagccccaggagtcgcccacccttccggtgtccaccgccaccgacagcagctactacaccaaccagcagcacccggcgggcggcggcggcggcgggggctcgccctacgcgcacatgggttcctaccagtaccaagccagcggcctcaacaacgtcccttactccgccaagagcagctatgacctgggctacaccgccgcctacacctcctacgctccctatggaaccagttcgtccccagccaacaacgagcctgagaaggaggaccttgagcctgaaattcggatagtgaacgggaagccaaagaaagtccggaaaccccgcaccatctactccagtttccagctggcggctcttcagcggcgtttccaaaagactcaatacttggccttgccggagcgagccgagctggcggcctctctgggcctcacccagactcaggtcaaaatctggttccagaaccgccggtccaagttcaagaagatgtggaaaagtggtgagatcccctcggagcagcaccctggggccagcgcttctccaccttgtgcttcgccgccagtctcagcgccggcctcctgggactttggtgtgccgcagcggatggcgggcggcggtggtccgggcagtggcggcagcggcgccggcagctcgggctccagcccgagcagcgcggcctcggcttttctgggcaactacccctggtaccaccagacctcgggatccgcctcacacctgcaggccacggcgccgctgctgcaccccactcagaccccgcagccgcatcaccaccaccaccatcacggcggcgggggcgccccggtgagcgcggggacgattttctaatgtacaagtccggactcagatcctactagtaggatctcgagggatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgagacctggaaaaacatggagcaatcacaagtagcaacacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 15 - DNA, 10939 bp, synthetic construct
  source 1..10939: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaattcactttgaattcaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcaccggtgccaccatgactggagtctttgacagtctagtggctgatatgcactcgacccagatcgccgcctccagcacgtaccaccagcaccagcagcccccgagcggcggcggcgccggcccgggtggcaacagcagcagcagcagcagcctccacaagccccaggagtcgcccacccttccggtgtccaccgccaccgacagcagctactacaccaaccagcagcacccggcgggcggcggcggcggcgggggctcgccctacgcgcacatgggttcctaccagtaccaagccagcggcctcaacaacgtcccttactccgccaagagcagctatgacctgggctacaccgccgcctacacctcctacgctccctatggaaccagttcgtccccagccaacaacgagcctgagaaggaggaccttgagcctgaaattcggatagtgaacgggaagccaaagaaagtccggaaaccccgcaccatctactccagtttccagctggcggctcttcagcggcgtttccaaaagactcaatacttggccttgccggagcgagccgagctggcggcctctctgggcctcacccagactcaggtcaaaatctggttccagaaccgccggtccaagttcaagaagatgtggaaaagtggtgagatcccctcggagcagcaccctggggccagcgcttctccaccttgtgcttcgccgccagtctcagcgccggcctcctgggactttggtgtgccgcagcggatggcgggcggcggtggtccgggcagtggcggcagcggcgccggcagctcgggctccagcccgagcagcgcggcctcggcttttctgggcaactacccctggtaccaccagacctcgggatccgcctcacacctgcaggccacggcgccgctgctgcaccccactcagaccccgcagccgcatcaccaccaccaccatcacggcggcgggggcgccccggtgagcgcggggacgattttctaatgtacaagtccggactcagatcctactagtaggatctcgagatgcagaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgagttttcgagggatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgagacctggaaaaacatggagcaatcacaagtagcaacacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 16 - DNA, 9460 bp, synthetic construct
  source 1..9460: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaattcactttgaattcaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcaccggtgccaccatgacaggagtgtttgacagaagagtcccaagcatccgatccagcgacttccaagctccgttcccgacgtccgccgccatgcaccacccgtctcaggaatcgccaactttgcccgagtcctcggccaccgattctgactactacagtcccgcgggggccgccccgcacggctactgctctcctacctctgcttcttatggcaaagcgctcaacccctaccagtaccagtaccacggcgtgaacggctccgcagccggctacccggccaaggcttatgccgactacggctaccacccctaccaccagtacggcggcgcctacaaccgcgtcccgagtgccaccagccagccagagaaagaagtggcggagccagaggtgaggatggtgaatggtaaaccaaagaaagttcgtaaacccaggactatttattccagctttcagctggccgctttacagagaaggtttcagaagactcagtacctcgccctgccagaacgcgcggagttggccgcctctctaggactgacgcaaacacaggtgaaaatctggtttcagaacaaaagatccaagatcaagaagatcatgaaaaacggggagatgcccccggagcacagtcccagctccagcgaccctatggcgtgtaactcgccacagtcaccagcggtgtgggagccccagggctcatcccgctctctcagccaccaccctcatgcccaccctccgacctccaaccagtcccccgcgtccagctacctggagaactcggcttcctggtacccaagcgcagccagctcaatcaattcccacctgccaccgccgggctccttgcagcacccgctggcactggcctccgggacgctttattagctcgagggatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgagacctggaaaaacatggagcaatcacaagtagcaacacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 17 - DNA, 681 bp, synthetic construct
  source 1..681: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc